(19) United States
(12) Patent Application Publication
(10) Pub. No.: US 2020/0157559 A1
(43) Pub. Date: May 21, 2020

(54) METHODS TO IMPROVE PLANT AGRONOMIC TRAIT USING BCS1L GENE AND GUIDE RNA/CAS ENDONUCLEASE SYSTEMS

(71) Applicants: PIONEER OVERSEAS CORPORATION, JOHNSTON, IA (US); SINOBIOWAY BIO-AGRICULTURE GROUP CO., LTD., BEIJING (CN)

(72) Inventors: GUIHUA LU, BEIJING (CN); GUANFAN MAO, BEIJING (CN); YANLONG SHI, BEIJING (CN); CHANGGUI WANG, BEIJING (CN); GUOKUI WANG, BEIJING (CN)

(73) Assignees: PIONEER OVERSEAS CORPORATION, JOHNSTON, IA (US); SINOBIOWAY BIO-AGRICULTURE GROUP CO., LTD., BEIJING (CN)

(21) Appl. No.: 16/621,359

(22) PCT Filed: Jun. 12, 2018
(86) PCT No.: PCT/CN2018/090728

§ 371 Date: Dec. 11, 2019

(30) Foreign Application Priority Data
Jun. 13, 2017 (CN) 201710441868.9

(51) Int. Cl. C12N 15/82; C07K 14/42; C12N 9/22

(52) U.S. Cl. CPC C12N 15/8273; C07K 14/42; C12N 2310/20; C12N 9/22; C12N 2810/10; C12N 15/8213

(57) ABSTRACT

Compositions and methods are provided for agronomic trait improvement through modifying a target sequence in the genome of a plant or plant cell. The methods and compositions employ a guide RNA/Cas endonuclease system to provide an effective system for modifying or altering target sites within a genomic region of a plant or plant cell to provide improvement in a desirable agronomic trait such as drought tolerance, yield and stress tolerance. Compositions and methods are also provided for editing a nucleotide sequence in the genome of a cell.

### FIELD

The field of the disclosure relates to plant molecular biology, in particular, relates to methods for modifying or altering the genome of a plant to improve abiotic stress tolerance, such as drought stress.

### BACKGROUND

Stresses to plants may be caused by both biotic and abiotic agents. For example, biotic causes of stress include infection with pathogen, insect feeding, and parasitism by another plant such as mistletoe. Abiotic stresses include, for example, excessive or insufficient available water, temperature extremes, and synthetic chemicals such as herbicides.

Abiotic stress is the primary cause of crop loss worldwide, causing average yield losses more than 50% for major crops (Boyer, J. S. (1982) Science 218:443-448; Bray, E. A. et al. (2000) In Biochemistry and Molecular Biology of Plants, edited by Buchannan, B. B. et al., Amer. Soc. Plant Biol., pp. 1158-1249).

Recombinant DNA technology has made it possible to insert foreign DNA sequences into the genome of an organism to over-expression or suppression the expression of some genes, thus improving abiotic stress tolerance such as drought tolerance and altering the organism's phenotype. One method for inserting or modifying a DNA sequence involves homologous DNA recombination by introducing a transgenic DNA sequence flanked by sequences homologous to the genomic target.

Site-specific recombination has potential for application across a wide range of biotechnology-related fields. Meganucleases, zinc finger nucleases (ZFNs), and transcription activator-like effector nucleases (TALENs) containing a DNA-binding domain and a DNA cleavage domain enable genome modification. Recent advances in application of clustered, regularly interspaced, short palindromic repeats (CRISPR) have illustrated a method of genome modification that may be as robust as the comparable systems (meganucleases, ZFNs, and TALENs).

The CRISPR system is composed of a protein component (Cas) and a guide RNA (gRNA) that targets the protein to a specific locus for endonucleolytic cleavage. This system has been successfully engineered to target specific loci for endonucleolytic cleavage of mammalian, zebrafish, drosophila, nematode, bacteria, yeast, and plant genomes.

### SUMMARY

The following embodiments are among those encompassed by the disclosure:

In one embodiment, the present disclosure includes a CRISPR-Cas construct comprising at least one heterologous regulatory sequence operably linked to gRNA, wherein the gRNA is targeted to a genomic region containing endogenous BCS1L gene and its promoter. Further the BCS1L gene encodes a polypeptide comprising an amino acid sequence that is at least 95% identical to SEQ ID NO: 8. The BCS1L gene comprises a polynucleotide with nucleotide sequence of SEQ ID NO: 6 or 7 or an allelic variant thereof comprising 1 to about 10 nucleotide changes. The BCS1L promoter comprises a polynucleotide with nucleotide sequence of SEQ ID NO: 9.

In another embodiment, the present disclosure includes a plant in which the expression or the activity of an endogenous BCS1L polypeptide is decreased, when compared to the expression or the activity of wild-type BCS1L polypeptide from a control plant, wherein the plant exhibits at least one phenotype selected from the group consisting of: increased grain yield, increased abiotic stress tolerance and increased biomass, compared to the control plant, wherein the expression or the activity of an endogenous BCS1L polypeptide is decreased through an introduced genetic modification. wherein the introduced modification comprises (a) introducing a DNA fragment or deleting a DNA fragment or replacing a DNA fragment or introducing (b) one or more nucleotide changes in the genomic region comprising the endogenous BCS1L gene and its promoter, wherein the modification results in decreasing the expression or the activity of the endogenous BCS1L polypeptide.

Further, A plant comprising a mutated BCS1L gene, wherein the expression or activity of the BCS1L polypeptide is decreased or eliminated in the plant, when compared to a control plant, and wherein the plant exhibits at least one phenotype selected from the group consisting of: increased grain yield, increased abiotic stress tolerance and increased biomass, compared to the control plant. The mutant BCS1L gene with nucleotide sequence of at least 95% sequence identity to SEQ ID NO: 6 or 7.

A plant comprising a mutated BCS1L gene which resulted the early termination of the coding sequence, wherein the plants exhibited drought tolerance compared to the control plant.

A plant comprising a mutated BCS1L promoter, wherein the expression or the activity of the BCS1L polypeptide is decreased in the plant, when compared to a control plant, and wherein the plant exhibits at least one phenotype selected from the group consisting of: increased grain yield, increased abiotic stress tolerance and increased biomass, compared to the control plant. The mutant BCS1L promoter with nucleotide sequence of at least 90% sequence identity to SEQ ID NO: 9. The plant exhibits an increase in abiotic stress tolerance, and the abiotic stress is drought stress.

In another embodiment, the present disclosure includes any of the plants of the disclosure, wherein the plant is selected from the group consisting of rice, maize, soybean, sunflower, sorghum, canola, wheat, alfalfa, cotton, barley, millet, sugar cane and switchgrass.

In another embodiment, methods of making a plant in which the expression or the activity of an endogenous BCS1L polypeptide is decreased through an introduced genetic modification, when compared to the expression or activity of wild-type BCS1L polypeptide from a control plant are provided, and wherein the plant exhibits at least one phenotype selected from the group consisting of: increased drought tolerance, increased grain yield, increased abiotic stress tolerance and increased biomass, compared to the control plant, wherein the method comprises the steps of (a) introducing a DNA fragment, deleting a DNA fragment or replacing a DNA fragment, or introducing (b) one or more nucleotide changes in the genomic region comprising the endogenous BCS1L gene and its promoter, wherein the modification is effective for decreasing the expression or the activity of the endogenous BCS1L polypeptide. The modification is introduced using zinc finger nuclease, Transcription Activator-Like Effector Nuclease (TALEN), CRISPR-Cas/Cpf1 or meganuclease. Further, the modification is introduced using CRISPR-Cas system.

In yet another embodiment, methods are provided for increasing drought tolerance in a plant, comprising: (a) introducing into a regenerable plant cell a construct to reduce the expression or activity of endogenous BCS1L polypeptide; (b) regenerating a modified plant from the regenerable plant cell after step (a); and (c) obtaining a progeny plant derived from the modified plant of step (b), wherein said progeny plant exhibits increased drought tolerance when compared to a control plant.

The said construct comprising: at least one heterologous regulatory sequence operably linked to gRNA, wherein the gRNA is targeted to BCS1L gene or its promoter.

The said gRNA is targeted to SEQ ID NO: 6, 7 or 9. The gRNA comprises the nucleotide sequence of SEQ ID NO: 10-30. If the gRNA showed the nucleotide sequence of SEQ ID NO: 13, the targeted site is between Chr5:29332310-29332802 in rice genome, wherein the genome edit results in nucleotide insertion, DNA fragment replacement, or deletion near Chr5:29332310-Chr:529332802 in rice genome, thus inducing the expression of the OsBCS1L prematurely terminate, or amino acid replacement or deletion. If the gRNA is the nucleotide sequence of SEQ ID NO: 14, the targeted site is between Chr5: 29332065-29332085 in rice genome, wherein the genome edit results in nucleotide insertion or replacement, or DNA fragment replacement or deletion near Chr5: 29332065-29332085 in rice genome, thus inducing the expression of the OsBCS1L prematurely terminate, translation shift or amino acid replacement or deletion.

In another embodiment, methods are provided for enhancing grain yield in a rice plant, when compared to a control plant, wherein the plant exhibits enhanced grain yield under stress conditions, the method comprising the step of decreasing the expression or the activity of the endogenous BCS1L gene or a heterologous BCS1L gene in the rice plant.

In another embodiment, the present disclosure concerns delivering the gRNAs/Cas9 enzyme complex into a cell, a plant, or a seed. The cell may be eukaryotic, e.g., a yeast, insect or plant cell; or prokaryotic, e.g., a bacterial cell.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows the schemetic of sgRNA distribution in the genome of rice OsBCS1L gene.

FIG. 2 shows an example of single sgRNA distribution in the genome of rice OsBCS1L gene.

FIG. 3 shows an example of two sgRNAs distribution in the genome of rice OsBCS1L gene.

FIG. 4 shows an alignment of mutation induced by CRISPR-Cas construct DP2317 in rice plant. The mutations were identified by PCR and sequencing. The reference sequence represents the unmodified locus with each target site underlined. The PAM sequence and expected site of cleavage are also indicated. Deletion, insertion or replacement is shown by a “−”, an italicized underlined nucleotide or bolded italicized nucleotide, respectively. The reference and mutations 1-14 of target site correspond to SEQ ID NO: 31-45, respectively.

FIG. 5 shows an alignment of mutation induced by CRISPR-Cas construct DP2354. The mutations were identified by PCR and sequencing. The reference sequence represents the unmodified locus with each target site underlined. The PAM sequence and expected site of cleavage are also indicated. Deletion, insertion or replacement is shown by a “−”, an italicized underlined nucleotide or bolded italicized nucleotide, respectively. The reference and mutations 1-15 of target site correspond to SEQ ID NO: 46-61, respectively.

FIG. 6 shows an alignment of mutation induced by CRISPR-Cas construct DP2420. The mutations were identified by PCR and sequencing. The reference sequence represents the unmodified locus with each target site underlined. The PAM sequence and expected site of cleavage are also indicated. Deletion, insertion or replacement is shown by a “−”, an italicized underlined nucleotide or bolded italicized nucleotide, respectively. The reference and mutations 1-7 of target site correspond to SEQ ID NO: 62-69, respectively.

TABLE 1
SEQ ID NOs for nucleotide and amino acid sequences
provided in the sequence listing

| | | SEQ ID NO: | SEQ ID NO: |
|---|---|---|---|
| Source species | Clone Designation | (Nucleotide) | (Amino Acid) |
| Zea May | Ubiquitin Promoter | 1 | n/a |
| Artificial | Nucleus localization | 2 | n/a |
| | sequence | | |
| Cauliflower mosaic | CaMV 3′UTR | 3 | n/a |
| virus | | | |
| Otyza sativa | rU6-Promoter | 4 | n/a |
| Artificial | gRNA scaffold | 5 | n/a |
| Otyza sativa | OsBCS1L gene | 6, 7 | 8 |
| Otyza sativa | OsBCS1L promoter | 9 | n/a |
| Artificial | gRNA | 10-30 | n/a |
| Otyza sativa | Mutation sequence | 31-69 | n/a |

The Sequence List contains the one-letter code for nucleotide sequences and the three-letter code for amino acid sequences as defined in conformity with the IUPAC-IUBMB standards described in Nucleic Acids Res. 13:3021-3030 (1985) and in the Biochemical J. 219 (No.2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants; reference to “a cell” includes one or more cells and equivalents thereof known to those skilled in the art, and so forth.

As used herein:

The term “OsBCS1L (mitochondrial chaperone BCS1 like protein)” refers to a rice polypeptide that confers drought sensitive phenotype when over-expression and is encoded by the rice gene locus LOC_Os05g51130.1. “BCS1L polypeptide” refers herein to the OsBCS1L polypeptide and its homologs from other organisms.

The OsBCS1L polypeptide (SEQ ID NO: 8) is encoded by the coding sequence (CDS) (SEQ ID NO: 7) or cloned nucleotide sequence (SEQ ID NO: 6) at rice gene locus LOC_Os05g51130.1. This polypeptide is annotated as “mitochondrial chaperone BCS1, putative, expressed” in TIGR.

The OsBCS1L promoter is shown in SEQ ID NO: 9.

The terms “monocot” and “monocotyledonous plant” are used interchangeably herein. A monocot of the current disclosure includes plants of the Gramineae family.

The terms “dicot” and “dicotyledonous plant” are used interchangeably herein. A dicot of the current disclosure includes the following families: Brassicaceae, Leguminosae, and Solanaceae.

The “Plant” includes reference to whole plants, plant organs, plant tissues, seeds and plant cells and progeny of the same. Plant cells include, without limitation, cells from seeds, suspension cultures, embryos, meristematic regions, callus tissues, leaves, roots, shoots, gametophytes, sporophytes, pollen, and microspores.

The “Progeny” comprises any subsequent generation of a plant.

The term “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar or nitrogen concentrations, or by the observation of the expression level of a gene or genes, or by agricultural observations such as osmotic stress tolerance or yield.

The “Agronomic characteristic” is a measurable parameter including but not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor and seedling emergence under low temperature stress.

The “Genome” as it applies to plant cells encompasses not only chromosomal DNA found within the nucleus, but also organelle DNA found within subcellular components (e.g., mitochondria, plastid) of the cell.

An “allele” is one of two or more alternative forms of a gene occupying a given locus on a chromosome. When the alleles present at a given locus on a pair of homologous chromosomes in a diploid plant are the same, that plant is homozygous at that locus. If the alleles present at a given locus on a pair of homologous chromosomes in a diploid plant differ, that plant is heterozygous at that locus. If a transgene is present on one of a pair of homologous chromosomes in a diploid plant, that plant is hemizygous at that locus.

The “Gene” refers to a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. A mutated plant is a plant comprising a mutated gene.

As “targeted mutation” is a mutation in a native gene that was made by altering a target sequence within the native gene using a method involving a double-strand-break-inducing agent that is capable of inducing a double-strand break in the DNA of the target sequence as disclosed herein of known in the art.

“Polynucleotide”, “nucleic acid sequence”, “nucleotide sequence”, and “nucleic acid fragment” are used interchangeably and refer to a polymer of RNA or DNA that is single- or double-stranded, optionally containing synthetic, non-natural or altered nucleotide bases. Nucleotides (usually found in their 5′-monophosphate form) are referred to by their single-letter designation as follows: “A” for adenylate or deoxyadenylate, “C” for cytidylate or deoxycytidylate, and “G” for guanylate or deoxyguanylate for RNA or DNA, respectively; “U” for uridylate; “T” for deoxythymidylate; “R” for purines (A or G); “Y” for pyrimidines (C or T); “K” for G or T; “H” for A or C or T; “I” for inosine; and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, and sulfation, gamma-carboxylation of glutamic acid residues, hydroxylation and ADP-ribosylation.

“Regulatory sequences” refer to nucleotide sequences located upstream (5′ non-coding sequences), within, or downstream (3′ non-coding sequences) of a coding sequence, and influencing the transcription, RNA processing or stability, or translation of the associated coding sequence. Regulatory sequences may include, but are not limited to, promoters, translation leader sequences, introns, and poly-adenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription of genes in plant cells whether or not its origin is from a plant cell.

“Tissue-specific promoter” and “tissue-preferred promoter” may refer to a promoter that is expressed predominantly but not necessarily exclusively in one tissue or organ, but that may also be expressed in one specific cell or cell type.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a CRISPR-Cas construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a CRISPR-Cas DNA construct) has been introduced.

“Transformation” as used herein refers to both stable transformation and transient transformation.

A “nuclear localization signal” is a signal peptide which direct the protein to the nucleus (Raikhel. (1992) Plant Phys. 100:1627-1632).

“CRISPR-associated genes” refers to nucleic acid sequences that encode polypeptide components of clustered regularly interspersed short palindromic repeats (CRISPR)-associated systems (Cas), and the genes are generally coupled, associated or close to or in the vicinity of flanking CRISPR loci. The terms “Cas gene”, “CRISPR-associated gene” are used interchangeably herein. Examples include, but are not limited to, Cas3 and Cas9, which encode endonucleases from the CRISPR type I and type II systems, respectively.

“Cas endonuclease” refers to a Cas protein encoded by a Cas gene, wherein said Cas protein is capable of introducing a double strand break into a DNA target sequence. The Cas endonuclease is guided by the guide polynucleotide to recognize and optionally introduce a double strand break at a specific target site into the genome of a cell.

“Guide RNA (gRNA)” refers to a crRNA (CRISPR RNA):tracrRNA fused hybrid RNA molecule encoded by a customizable DNA element that, generally, comprises a copy of a spacer sequence which is complementary to the protospacer sequence of the genomic target site, and a binding domain for an associated-Cas endonuclease of the CRISPR complex.

“Guide polynucleotide” refers to a polynucleotide sequence that can form a complex with a Cas endonuclease and enables the Cas endonuclease to recognize and optionally cleave a DNA target site. The guide polynucleotide can be comprised of a single molecule or a double molecule.

The term “guide polynucleotide/Cas endonuclease system” refers to a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA, but only if the correct protospacer-adjacent motif (PAM) is approximately oriented at the 3′ end of the target sequence.

“Genomic target site” refers to a protospacer and a protospacer adjacent motif (PAM) located in a host genome selected for targeted mutation and/or double-strand break.

“Protospacer” refers to a short DNA sequence (12 to 40 bp) that can be targeted for mutation, and/or double-strand break, mediated by enzymatic cleavage with a CRISPR system endonuclease guided by complementary base-pairing with the spacer sequence in the crRNA or sgRNA.

“Protospacer adjacent motif (PAM)” includes a 3 to 8 bp sequence immediately adjacent to the protospacer sequence in the genomic target site.

CRISPR loci (Clustered Regularly Interspaced Short Palindromic Repeats) (also known as SPIDRs-SPacer Interspersed Direct Repeats) constitute a family of recently described DNA loci. CRISPR loci consist of short and highly conserved DNA repeats (typically 24 to 40 bp, repeated from 1 to 140 times-also referred to as CRISPR-repeats) which are partially palindromic. The repeated sequences (usually specific to a species) are interspaced by variable sequences of constant length (typically 20 to 58 bp by depending on the CRISPR locus (WO2007/025097 published Mar. 1, 2007).

Endonucleases are enzymes that cleave the phosphodiester bond within a polynucleotide chain, and include restriction endonucleases that cleave DNA at specific sites without damaging the bases. Restriction endonucleases include Type I, Type II, Type III, and Type IV endonucleases, which further include subtypes. In the Type I and Type III systems, both the methylase and restriction activities are contained in a single complex. Endonucleases also include meganucleases, also known as homing endonucleases (HEases), which like restriction endonucleases, bind and cut at a specific recognition site, however the recognition sites for meganucleases are typically longer, about 18 bp or more (patent application WO-PCT PCT/US12/30061 filed on Mar. 22, 2012). Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates.

TAL effector nucleases are a new class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, Foki. The unique, modular TAL effector DNA binding domain allows for the design of proteins with potentially any given DNA recognition specificity (Miller et al. (2011) Nature Biotechnology 29:143-148). Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs consist of an engineered DNA-binding zinc finger domain linked to a non-specific endonuclease domain, for example nuclease domain from a Type lis endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3 finger domain recognized a sequence of 9 contiguous nucleotides, with a dimerization requirement of the nuclease, two sets of zinc finger triplets are used to bind an 18 nucleotide recognition sequence.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a plant cell at which a double-strand break is induced in the plant cell genome by a Cas endonuclease. The target site can be an endogenous site in the plant genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeable herein to refer to a target sequence that is endogenous or native to the genome of a plant and is at the endogenous or native position of that target sequence in the genome of the plant.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

“Drought” refers to a decrease in water availability to a plant that, especially when prolonged or when occurring during critical growth periods, can cause damage to the plant or prevent its successful growth (e.g., limiting plant growth or seed yield).

“Drought tolerance” reflects a plant's ability to survive under drought without exhibiting substantial physiological or physical deterioration, and/or its ability to recover when water is restored following a period of drought.

“Increased drought tolerance” of a plant is measured relative to a reference or control plant, and reflects ability of the plant to survive under drought conditions with less physiological or physical deterioration than a reference or control plant grown under similar drought conditions, or ability of the plant to recover more substantially and/or more quickly than would a control plant when water is restored following a period of drought.

“Environmental conditions” refer to conditions under which the plant is grown, such as the availability of water, availability of nutrients, or the presence of insects or disease.

“Paraquat” (1,1-dimethyl-4,4-bipyridinium dichloride), is a foliar-applied and non-selective bipyridinium herbicides, and causes photooxidative stress which further cause damage to plant or prevent its successful growth.

“Paraquat tolerance” is a trait of a plant, reflects the ability to survive and/or grow better when treated with Paraquat solution, compared to a reference or control plant.

“Increased paraquat tolerance” of a plant is measured relative to a reference or control plant, and reflects ability of the plant to survive with less physiological or physical deterioration than a reference or control plant after treated with paraquat solution. In general, tolerance to relative low level of paraquat can be used as a marker of abiotic stress tolerance, such as drought tolerance.

“Oxidative stress” reflects an imbalance between the systemic manifestation of reactive oxygen species and a biological system's ability to readily detoxify the reactive intermediates or to repair the resulting damage. Disturbances in the normal redox state of cells can cause toxic effects through the production of peroxides and free radicals that damage all components of the cell, including proteins, lipids, and DNA.

“Percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence =number of identical positions between query and subject sequences/total number of positions of query sequence ×100).

### CRISPR-Cas Constructs:

A CRISPR-Cas construct comprising: a polynucleotide encoding a CRISPR enzyme, a polynucleotide encoding nuclear localization signal and at least one heterologous regulatory sequence operably linked to gRNA, wherein the gRNA is targeted to the genomic region containing endogenous BCS1L gene and its promoter.

Further the gRNA is targeted to the genomic region containing the polynucleotide with nucleotide sequence of SEQ ID NO: 6, 7 or 9.

### Regulatory Sequences:

A regulatory sequence may be a promoter, enhancer, 5′UTR, or 3′UTR.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High-level, constitutive expression of the candidate gene under control of the 35S or UBI promoter may have pleiotropic effects, although candidate gene efficacy may be estimated when driven by a constitutive promoter. Use of tissue-specific and/or stress-induced promoters may eliminate undesirable effects but retain the ability to enhance drought tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally-regulated promoter is a DNA sequence which regulates the expression of a DNA sequence selectively in the cells/tissues of a plant, such as in those cells/tissues critical to tassel development, seed set, or both, and which usually limits the expression of such a DNA sequence to the developmental period of interest (e.g. tassel development or seed maturation) in the plant. Any identifiable promoter which causes the desired temporal and spatial expression may be used in the methods of the present disclosure.

For the expression of a polynucleotide in developing seed tissue, promoters of particular interest include seed-preferred promoters, particularly early kernel/embryo promoters and late kernel/embryo promoters. Kernel development post-pollination is divided into approximately three primary phases. The lag phase of kernel growth occurs from about 0 to 10-12 DAP. During this phase the kernel is not growing significantly in mass, but rather important events are being carried out that will determine kernel vitality (e.g., number of cells established). The linear grain fill stage begins at about 10-12 DAP and continues to about 40 DAP. During this stage of kernel development, the kernel attains almost all of its final mass, and various storage products (i.e., starch, protein, oil) are produced. Finally, the maturation phase occurs from about 40 DAP to harvest. During this phase of kernel development, the kernel becomes quiescent and begins to dry down in preparation for a long period of dormancy prior to germination. As defined herein “early kernel/embryo promoters” are promoters that drive expression principally in developing seed during the lag phase of development (i.e., from about 0 to about 12 DAP). “Late kernel/embryo promoters”, as defined herein, drive expression principally in developing seed from about 12 DAP through maturation. There may be some overlap in the window of expression. The choice of the promoter will depend on the ABA-associated sequence utilized and the phenotype desired.

Early kernel/embryo promoters include, for example, Cim1 that is active 5 DAP in particular tissues (WO 00/11177), which is herein incorporated by reference. Other early kernel/embryo promoters include the seed-preferred promoters end1 which is active 7-10 DAP, and end2, which is active 9-14 DAP in the whole kernel and active 10 DAP in the endosperm and pericarp (WO 00/12733), herein incorporated by reference. Additional early kernel/embryo promoters that find use in certain methods of the present disclosure include the seed-preferred promoter Itp2 (U.S. Pat. No. 5,525,716); maize Zm40 promoter (U.S. Pat. No. 6,403,862); maize nuc1c (U.S. Pat. No. 6,407,315); maize ckx1-2 promoter (U.S. Pat. No. 6,921,815 and US Patent Application Publication Number 2006/0037103); maize led promoter (U.S. Pat. No. 7,122,658); maize ESR promoter (U.S. Pat. No. 7,276,596); maize ZAP promoter (U.S. Patent Application Publication Numbers 20040025206 and 20070136891); maize promoter eepl (U.S. Patent Application Publication Number 20070169226); and maize promoter ADF4 (U.S. Patent Application No. 60/963,878, filed 7 Aug. 2007).

Promoters for use in certain embodiments of the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays; root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664).

An intron sequence can be added to the 5′ untranslated region, the protein-coding region or the 3′ untranslated region to increase the amount of the mature message RNA that accumulates in the cytosol. Inclusion of a spliceable intron in the transcription unit in both plant and animal expression constructs has been shown to increase gene expression at both the mRNA and protein levels up to 1000-fold (Buchman and Berg. (1988) Mol. Cell Biol. 8:4395-4405; Callis et al. (1987) Genes Dev. 1:1183-1200).

An enhancer or enhancer element refers to a cis-acting transcriptional regulatory element, a.k.a. cis-element, which confers an aspect of the overall expression pattern, but is usually insufficient alone to drive transcription, of an operably linked polynucleotide sequence. An isolated enhancer element may be fused to a promoter to produce a chimeric promoter cis-element, which confers an aspect of the overall modulation of gene expression. Enhancers are known in the art and include the SV40 enhancer region, the CaMV 35S enhancer element, and the like. Some enhancers are also known to alter normal regulatory element expression patterns, for example, by causing a regulatory element to be expressed constitutively when without the enhancer, the same regulatory element is expressed only in one specific tissue or a few specific tissues. Duplicating the upstream region of the CaMV35S promoter has been shown to increase expression by approximately tenfold (Kay, R. et al., (1987) Science 236: 1299-1302).

### Compositions:

A composition of the present disclosure is a plant in which the expression or the activity of an endogenous BCS1L polypeptide is decreased, when compared to the expression or the activity of wild-type BCS1L polypeptide from a control plant, wherein the plant exhibits at least one phenotype selected from the group consisting of: increased drought tolerance, increased grain yield, increased abiotic stress tolerance and increased biomass, compared to the control plant, wherein the expression or the activity of an endogenous BCS1L polypeptide is decreased through an introduced genetic modification. wherein the modification comprises (a) introducing a DNA fragment or deleting a DNA fragment or replacing a DNA fragment or introducing (b) one or more nucleotide changes in the genomic region comprising the endogenous BCS1L gene and its promoter, wherein the change is effective for decreasing the expression or the activity of the endogenous BCS1L polypeptide.

A composition of the present disclosure is a plant comprising a modified BCS1L gene, or a plant in which BCS1L gene promoter is modified. Compositions also include any progeny of the plant, and any seed obtained from the plant or its progeny, wherein the progeny or seed comprises within its genome the modified BCS1L gene or promoter. Progeny includes subsequent generations obtained by self-pollination or out-crossing of a plant. Progeny also includes hybrids and inbreds.

In hybrid seed propagated crops, mature modified plants can be self-pollinated to produce a homozygous inbred plant. The inbred plant produces seed containing the modified BCS1L gene or promoter. These seeds can be grown to produce plants that would exhibit an altered agronomic characteristic (e.g., an increased agronomic characteristic optionally under water limiting conditions), or used in a breeding program to produce hybrid seed, which can be grown to produce plants that would exhibit such an altered agronomic characteristic. The seeds may be maize seeds or rice seeds.

The plant may be a monocotyledonous or dicotyledonous plant, for example, a rice or maize or soybean plant, such as a maize hybrid plant or a maize inbred plant. The plant may also be sunflower, sorghum, canola, wheat, alfalfa, cotton, barley, millet, sugar cane or switchgrass.

The CRISPR-Cas construct may be stably integrated into the genome of the plant. The modification in the gene or promoter may be stably inherited in the plant.

Particular embodiments include but are not limited to the following:

1. A modified plant (for example, a rice or maize or soybean plant) comprising (a) a modified polynucleotide with nucleotide sequence of at least 85% sequence identity to SEQ ID NO: 6; (b) a modified polynucleotide with nucleotide sequence of at least 85% sequence identity to SEQ ID NO: 7; or (c) the full complement of the nucleotide sequence of (a) or (b), wherein the plant exhibits enhanced drought tolerance.

2. A modified plant (for example, a rice or maize or soybean plant) comprising (a) a modified polynucleotide with nucleotide sequence of at least 95% sequence identity to SEQ ID NO: 6; (b) a modified polynucleotide with nucleotide sequence of at least 95% sequence identity to SEQ ID NO: 7; or (c) the full complement of the nucleotide sequence of (a) or (b), wherein the plant exhibits enhanced drought tolerance.

3. A modified plant, wherein expression of the BCS1L gene is decreased in the plant, when compared to a control plant, and wherein the plant exhibits at least one phenotype selected from the group consisting of: increased grain yield, increased abiotic stress tolerance and increased biomass, compared to the control plant, the plant exhibits an increase in abiotic stress tolerance, and the abiotic stress is drought stress.

4. Any progeny of the above plants in embodiment 1-3, any seeds of the above plants in embodiment 1-3, any seeds of progeny of the above plants in embodiment 1-3, and cells from any of the above plants in embodiment 1-3 and progeny thereof.

In any of the foregoing embodiment 1-4 or other embodiments, the alteration of at least one agronomic characteristic is an increase.

In any of the foregoing embodiment 1-4 or other embodiments, the plant may exhibit the alteration of at least one agronomic characteristic when compared, under water limiting conditions, to a control plant.

The Examples below describe some representative protocols and techniques for simulating drought conditions and/or evaluating drought tolerance; and simulating oxidative conditions.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to yield loss exhibited by a control or reference plant).

Parameters such as recovery degree, survival rate, paraquat tolerance rate, gene expression level, water use efficiency, level or activity of an encoded protein, and others are typically presented with reference to a control cell or control plant.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration. One of ordinary skill in the art would readily recognize a suitable control or reference plant to be utilized when assessing or measuring an agronomic characteristic or phenotype of a transgenic plant using compositions or methods as described herein. For example, by way of non-limiting illustrations:

1. Progeny of a modified plant which is hemizygous with respect to a modified polynucleotide, such that the progeny are segregating into plants either comprising or not comprising the modified polynucleotide: the progeny comprising the modified polynucleotide would be typically measured relative to the progeny not comprising the modified polynucleotide. The progeny not comprising the modified polynucleotide is the control or reference plant.

2. Introgression of a modified polynucleotide into an inbred line, such as in rice and maize, or into a variety, such as in soybean: the introgressed line would typically be measured relative to the parent inbred or variety line (i.e., the parent inbred or variety line is the control or reference plant).

3. Two hybrid lines, wherein the first hybrid line is produced from two parent inbred lines, and the second hybrid line is produced from the same two parent inbred lines except that one of the parent inbred lines contains a modified polynucleotide: the second hybrid line would typically be measured relative to the first hybrid line (i.e., the first hybrid line is the control or reference plant).

4. A plant comprising a modified polynucleotide: the plant may be assessed or measured relative to a control plant not comprising the modified polynucleotide but otherwise having a comparable genetic background to the plant (e.g., sharing at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99%, or 100% sequence identity of nuclear genetic material compared to the plant comprising the modified polynucleotide.

A control plant or plant cell may comprise, for example: (a) a wild-type (WT) plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimulus that would induce expression of the gene of interest or (e) the subject plant or plant cell itself, under conditions in which the gene of interest is not expressed. A control may comprise numerous individuals representing one or more of the categories above; for example, a collection of the non-transformed segregants of category “c” is often referred to as a bulk null.

In this disclosure, ZH11-TC, DP0158 and genome editing negative indicate control plants, ZH11-TC represents rice plants generated from tissue cultured Zhonghua 11, DP0158 represents rice plants transformed with empty vector of DP0158, genome editing negative represents rice plants which were transformed with the CRISPR-Cas constructs but no modification produced at the target sites.

### Methods:

Methods include but are not limited to methods for modifying or altering the host endogenous genomic gene, methods for altering the expression and/or activity of endogenous polypeptide, methods for increasing drought tolerance in a plant, methods for evaluating drought tolerance in a plant, methods for increasing paraquat tolerance, methods for altering an agronomic characteristic in a plant, methods for determining an alteration of an agronomic characteristic in a plant, and methods for producing seed.

Methods include but are not limited to the following:

Methods are provided for genome modification of a target sequence in the genome of a plant or plant cell, for selecting plants, for gene editing, and for inserting a polynucleotide of interest into the genome of a plant. The methods employ a guide RNA/Cas endonuclease system, wherein the Cas endonuclease is guided by the guide RNA to recognize and optionally introduce a double strand break at a specific target site into the genome of a cell. The guide RNA/Cas endonuclease system provides for an effective system for modifying target sites within the genome of a plant, plant cell or seed. Further provided are methods and compositions employing a guide polynucleotide/Cas endonuclease system to provide an effective system for modifying target sites within the genome of a cell and for editing a nucleotide sequence in the genome of a cell. Once a genomic target site is identified, a variety of methods can be employed to further modify the target sites such that they contain a variety of polynucleotides of interest.

In one embodiment, a method for modifying a target site in the genome of a plant cell, comprises introducing a guide RNA and a Cas endonuclease into said plant, wherein said guide RNA and Cas endonuclease are capable of forming a complex that enables the Cas endonuclease to introduce a double strand break at said target site.

Further provided is a method for modifying a target site in the genome of a plant cell, the method comprising: a) introducing into a plant cell a guide RNA and a Cas endonuclease, wherein said guide RNA and Cas endonuclease are capable of forming a complex that enables the Cas endonuclease to introduce a double strand break at said target site; and, b) identifying at least one plant cell that has a modification at said target site, wherein the modification includes at least one deletion, insertion or substitution of one or more nucleotides in said target site.

Proteins may be altered in various ways including amino acid substitution, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

A method for editing a nucleotide sequence in the genome of a cell, the method comprising introducing a guide polynucleotide, a Cas endonuclease, and optionally a polynucleotide modification template, into a cell, wherein said guide RNA and Cas endonuclease are capable of forming a complex that enables the Cas endonuclease to introduce a double strand break at target site in the genome at said cell, wherein said polynucleotide modification template comprises at least one nucleotide modification of said nucleotide sequence. The nucleotide sequence in the genome of a cell is selected from the group consisting of a promoter sequence, a terminator sequence, a regulatory element sequence, a splice site, a coding sequence, a polyubiquitination site, an intron site and an intron enhancing motif.

A method for editing a promoter sequence in the genome of a cell, the methods comprising introducing a guide polynucleotide, a polynucleotide modification template and at least one Cas endonuclease into a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence.

A method for transforming a cell comprising transforming a cell with any one or more of the CRISPR-Cas vector of the present disclosure, wherein, in particular embodiments, the cell is eukaryotic cell, e.g., a yeast, insect or plant cell; or prokaryotic cell, e.g., a bacterial cell.

A method for producing a modified plant comprising transforming a plant cell with any of the CRISPR-Cas construct of the present disclosure and regenerating a modified plant from the transformed plant cell, wherein, the modified plant and the modified seed obtained by this method may be used in other methods of the present disclosure.

A method for altering the expression level of a polypeptide of the disclosure in a plant comprising: (a) transforming a regenerable plant cell with a CRISPR-Cas construct of the present disclosure; and (b) regenerating a modified plant from the regenerable plant cell after step (a), wherein the plant gene were edited; and (c) growing the transformed plant, wherein the expression of the CRISPR-Cas construct results in production of altered levels of the polypeptide of the disclosure in the transformed plant.

A method of making a plant in which the expression or the activity of an endogenous BCS1L polypeptide is decreased through an introduced genetic modification, when compared to the expression or activity of wild-type BCS1L polypeptide from a control plant, and wherein the plant exhibits at least one phenotype selected from the group consisting of:

increased drought tolerance, increased grain yield, increased abiotic stress tolerance and increased biomass, compared to the control plant, wherein the method comprises the steps of (i) introducing a DNA fragment, deleting a DNA fragment or replacing a DNA fragment, or introducing (ii) one or more nucleotide changes in the genomic region comprising the endogenous BCS1L gene and its promoter, wherein the change is effective for decreasing the expression or the activity of the endogenous BCS1L polypeptide.

A method of making a plant in which the expression or the activity of an endogenous OsBCS1L polypeptide is decreased, when compared to the activity of wild-type OsBCS1L polypeptide from a control plant, and wherein the plant exhibits at least one phenotype selected from the group consisting of: increased drought tolerance, increased grain yield, increased abiotic stress tolerance and increased biomass, compared to the control plant, wherein the method comprises the steps of (i) introducing a DNA fragment, deleting a DNA fragment or replacing a DNA fragment, or introducing (ii) one or more nucleotide changes in the genomic region near Chr5:29332310-29332330, Chr5:29332065-29332085 or Chr5:29332310-29332802, wherein the change is effective for decreasing the expression or the activity of the endogenous OsBCS1L polypeptide.

A method of increasing drought tolerance and/or paraquat tolerance in a plant, comprising: (a) introducing into a regenerable plant cell a CRISPR-Cas construct comprising a polynucleotide encoding a CRISPR enzyme, a polynucleotide encoding nuclear localization signal and at least one heterologous regulatory sequence operably linked to gRNA, wherein the gRNA is targeted to the genomic region comprising BCS1L gene and its promoter; (b) obtaining a progeny plant derived from said modified plant, wherein the progeny plant comprises in its genome the modified BCS1L gene or its promoter and exhibits increased drought tolerance when compared to a control plant.

A method of increasing drought tolerance and/or paraquat tolerance in a plant, comprising: (a) introducing into a regenerable plant cell a CRISPR-Cas construct comprising a polynucleotide encoding a CRISPR enzyme, a polynucleotide encoding nuclear localization signal and at least one heterologous regulatory sequence operably linked to gRNA, wherein the gRNA is targeted to SEQ ID NO: 6, 7 or 9; (b) obtaining a progeny plant derived from said modified plant, wherein the progeny plant comprises in its genome the modified OsBCS1L gene and exhibits increased drought tolerance when compared to a control plant.

A method of producing seed comprising any of the preceding methods, and further comprising obtaining seeds from said progeny plant, wherein said seeds comprise in their genome the modified BCS1L gene or its promoter.

In any of the preceding methods or any other embodiments of methods of the present disclosure, in said introducing step, the said regenerable plant cell may comprise a callus cell, an embryogenic callus cell, a gametic cell, a meristematic cell, or a cell of an immature embryo. The regenerable plant cells may derive from an inbred maize plant or rice.

In any of the preceding methods or any other embodiments of methods of the present disclosure, said regenerating step may comprise the following: (i) culturing said transformed plant cells in a medium comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the step of determining an alteration of an agronomic characteristic in a modified plant, if applicable, may comprise determining whether the modified plant exhibits an alteration of at least one agronomic characteristic when compared, under varying environmental conditions, to a control plant or the wild-type plant.

In any of the preceding methods or any other embodiments of methods of the present disclosure, the step of determining an alteration of an agronomic characteristic in a progeny plant, if applicable, may comprise determining whether the progeny plant exhibits an alteration of at least one agronomic characteristic when compared, under varying environmental conditions, to a control plant or the wild-type plant.

In any of the preceding methods or any other embodiments of methods of the present disclosure, the plant may exhibit the alteration of at least one agronomic characteristic when compared, under water limiting conditions to a control plant.

The introduction of CRSIPR-Cas construct of the present disclosure into plants may be carried out by any suitable technique, including but not limited to vector-mediated DNA transfer, bombardment, or Agrobacterium-mediated transformation, biolistic particle bombardment. Techniques for plant transformation and regeneration have been described in International Patent Publication WO 2009/006276, the contents of which are herein incorporated by reference.

The development or regeneration of modified plants is well known in the art. The regenerated plants may be self-pollinated to provide homozygous modified plants. Otherwise, pollen obtained from the regenerated plants is crossed to seed-grown plants of agronomically important lines. Conversely, pollen from plants of these important lines is used to pollinate regenerated plants.

### EXAMPLES

The present disclosure is further illustrated in the following examples, in which parts and percentages are by weight and degrees are Celsius, unless otherwise stated. It should be understood that these examples, while indicating embodiments of the disclosure, are given by way of illustration only. From the above discussion and these examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

### Example 1

### Design of sgRNA Sequence

Target genomic sequences are analyzed using available tools to generate candidate sgRNA sequences. The sgRNA sequences can also be generated by other web-tools including, but not limited to, the web site http://cbi.hzau.edu.cn/crispr/ and CRISPR-PLANT, available online.

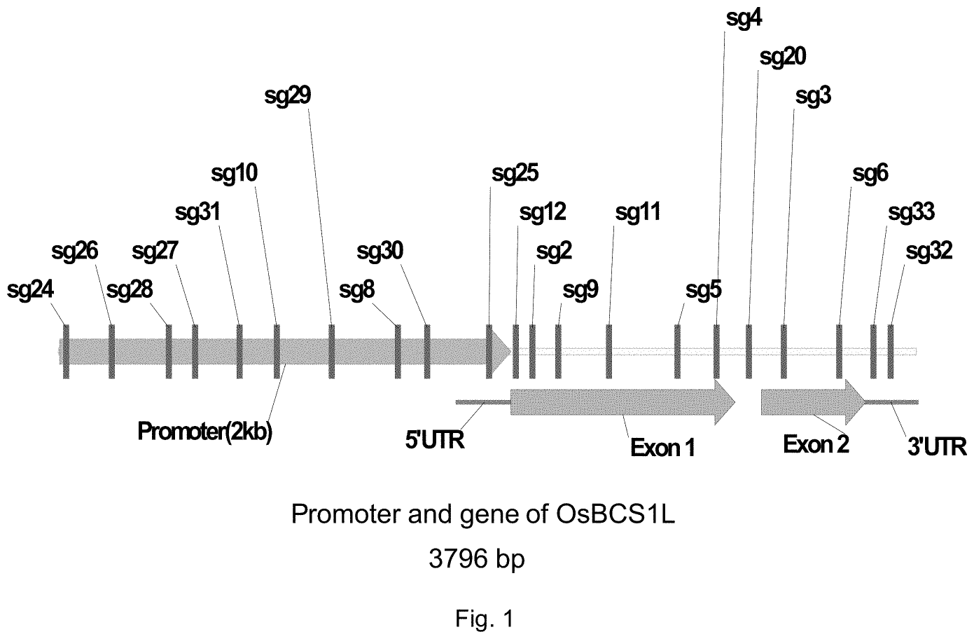

In this application, the OsBCS1L promoter and gene sequence (SEQ ID NO: 9 and SEQ ID NO: 10) was analyzed to generate the sgRNA sequences. The OsBCS1L promoter and gene sequence includes promoter, exon, intron, 5′-UTR, and 3′-UTR, and many sgRNA sequences were generated. 21 sgRNA sequences were selected and the distributions were shown in FIG. 1. The sgRNA sequences were listed in SEQ ID NO: 10-30.

### Example 2

### Construction of CRIPSR-Cas Constructs for OsBCS1L Gene

In the CRISPR-Cas9 system, maize Ubi promoter (SEQ ID NO: 1) drives the optimized coding sequence (SEQ ID NO: 2) of Cas9 protein; CaMV35S 3′-UTR (SEQ ID NO: 3) improves the expression level of Cas9 protein; and rice U6 promoter (SEQ ID NO: 4) drives the expression of gRNA (gRNA scaffold, SEQ ID NO: 5).

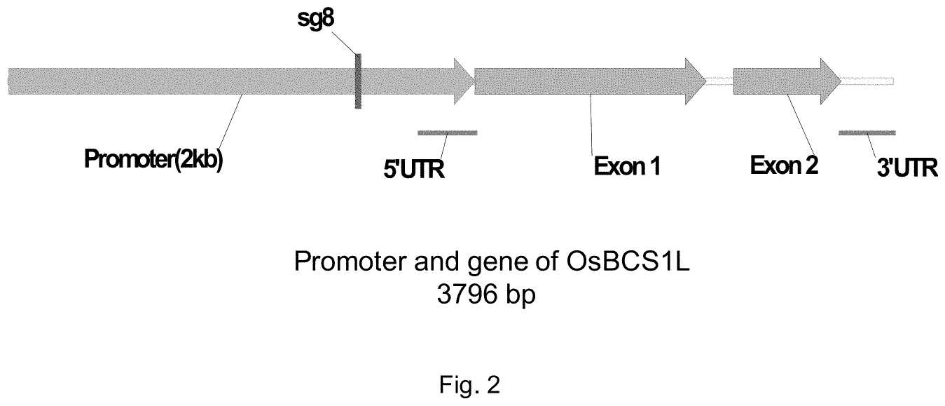

One sgRNA can be used to make the genome editing construct (FIG. 2.); the sgRNA can be selected from any region of the fragment such as promoter, exon, intron and UTR. The single sgRNA can guide the Cas9 enzyme to the target region and generate the double strand break at the target DNA sequence, non-homologous end-joining (NHEJ) repairing mechanism and homology directed repair (HDR) will be triggered, and it often induces random insertion, deletion and substitution at the target site. This edit, for example, can remove an expression element in the promoter region of OsBCS1L to reduce the mRNA levels or can result in a structural change in the OsBCS1L polypeptide that may result in reduced activity of the OsBCS1L protein.

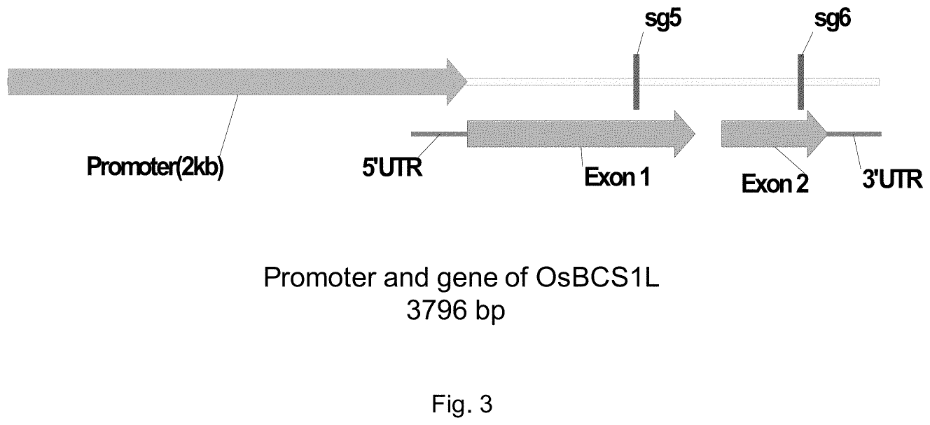

Two sgRNAs can be used to make the genome editing construct (FIG. 3.); two or more sgRNAs can be selected from any region of the fragment such as promoter, exon, intron and UTR. This construct can lead to fragment deletion, point mutation (small insertion, deletion and substitution).

Table 2 showed the primer sequence, target position and the specific strand. For the construct DP2317 and DP2354, one sgRNA was used. The target primers first annealed to form short double strand fragment, then the fragment was inserted in pHSG396GW-URS-UC-mpCas9&U6-DsRed (improved vector from VK005-01, which was bought from Beijing Veiwsolid Biotech company). The elements in the cloning vector pHSG396GW-URS-UC-mpCas9&U6-DsRed were shown in SEQ ID NO: 1, SEQ ID NO: 2, SEQ ID NO: 3, SEQ ID NO: 4 and SEQ ID NO: 5. After confirming the nucleotide sequence of gRNA fragment, the gRNA fragment was ligated with the expression vector PCAMBIA1300DsRed-GW-Adv.ccdB. For the construct having two sgRNAs, the different primers should first anneal to form the double strand fragments, then the two gRNA fragments stacked together and inserted in the cloning vector, and then were inserted in the expression vector to form DP2420, DP3090 and DP3091. The expected cleaving site was shown in FIG. 1.

The sgRNA(s) in the construct DP2317, DP2354 and DP2420 targets to the genomic region containing OsBCS1L gene, and the sgRNAs in the construct DP3090 and DP3091 target to the genomic region containing OsBCS1L promoter.

TABLE 2
Primers for constructing CRISPR/Cas9 constructs
for OsBCS1L gene and promoter editing

| Con- | Target | | | | SEQ |
|---|---|---|---|---|---|
| struct | site | | Strand | Target site | ID |
| ID | ID | Target position | (+/−) | primer sequence | NO: |
| DP2317 | gRNA6 | Chr5:29332065- | − | 5′-AAAAGATGCCAGCGAGACCA-3′ | 14 |
| | | 29332085 | | | |
| DP2354 | gRNA5 | Chr5:29332782- | + | 5′-TCCCTGGTGGACCATACAGC-3′ | 13 |
| | | 29332802 | | | |
| DP2420 | gRNA3 | Chr5:29332310- | − | 5′-GATCAAGCACTTATGCGGCC-3′ | 11 |
| | | 29332330 | | | |
| | gRNA5 | Chr5:29332782- | + | 5′-TCCCTGGTGGACCATACAGC-3′ | 13 |
| | | 29332802 | | | |
| DP3090 | gRNA8 | Chr5:29334022- | − | 5′-TTCAGTAAAGAAGATACGCT-3′ | 15 |
| | | 29334041 | | | |
| | gRNA25 | Chr5:29333617- | + | 5′-AACAGTAGTGTTGAATGGGG-3′ | 22 |
| | | 29333636 | | | |
| DP3091 | gRNA10 | Chr5:29334558- | − | 5′-GATCAACAATTTGACCCCTT-3′ | 17 |
| | | 29334577 | | | |
| | gRNA25 | Chr5:29333617- | + | 5′-AACAGTAGTGTTGAATGGGG-3′ | 22 |
| | | 29333636 | | | |

### Example 3

### Transformation to get Genome Edited Rice

The CRISPR-Cas9 constructs for OsBCS1L gene were transformed into the Zhonghua 11 (Oryza sativa L.) by Agrobacteria-mediated method as described by Lin and Zhang ((2005) Plant Cell Rep. 23:540-547). The transformed seedlings (TO) generated in transformation laboratory were first validated by PCR and sequencing, and then were planted in the field to get T1 seeds. The T1 and T2 seeds are stored at cold room (4° C.).

### Example 4

### Identification the Cleavage Sites and the Modifications of OsBCS1L Gene in Rice Plants

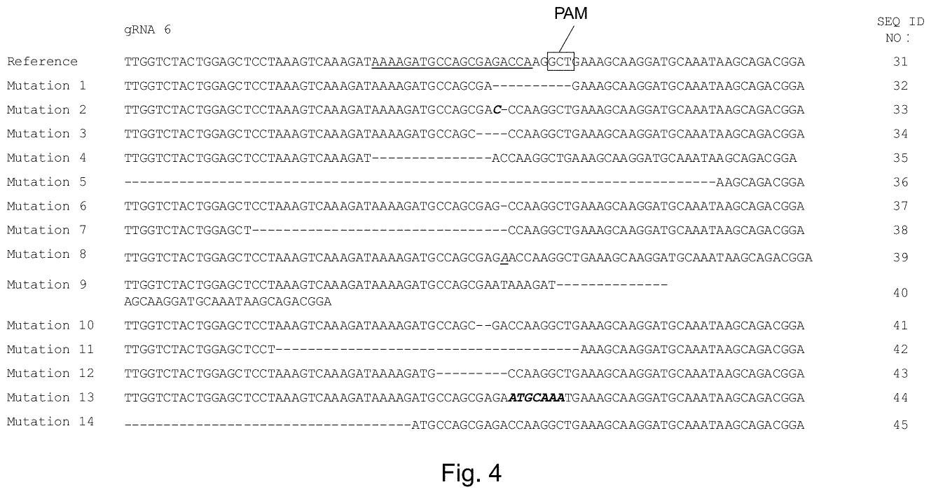

The primers were designed to amplified the target sequence near the genome editing target sites using the genome DNA of the transformed seedlings as template. The amplified target sequences were sequenced to confirm the editing results, which were shown in FIGS. 4, 5 and 6. Modifications such as insertion of at least one nucleotide, deletion of at least one nucleotide, replacement of at least one nucleotide were produced, which resulted the early termination of the coding sequence, translation shift and/or deletion of at least one amino acid residues.

As shown in FIG. 4, there are about 14 modifications produced at the expect sites in DP2317 rice plants. Seven mutants resulted in translation shift, but the translations were not stopped at the original stop code site; 5 mutants resulted in early stops of the ORF and further resulted in 443 to 454 amino acid residues in length; 1 mutant resulted in deletion of 9 nucleotides and deletion of 3 amino acid residues; and 1 mutant resulted in replacement of 3 amino acid residues.

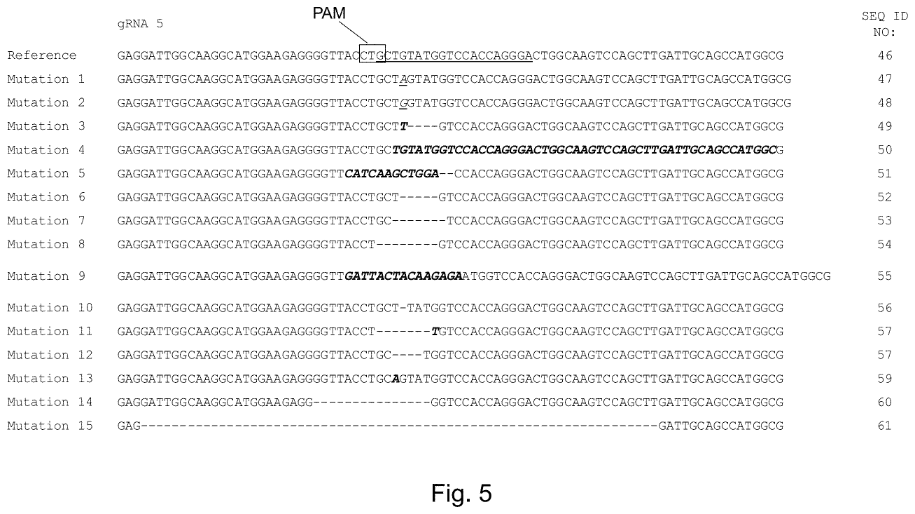

As shown in FIG. 5, there are about 12 modifications produced at the expect sites in DP2354 rice plants. All these 12 mutants resulted in early termination of translation. The translated polypeptides have 244 to 284 amino acid residues in length.

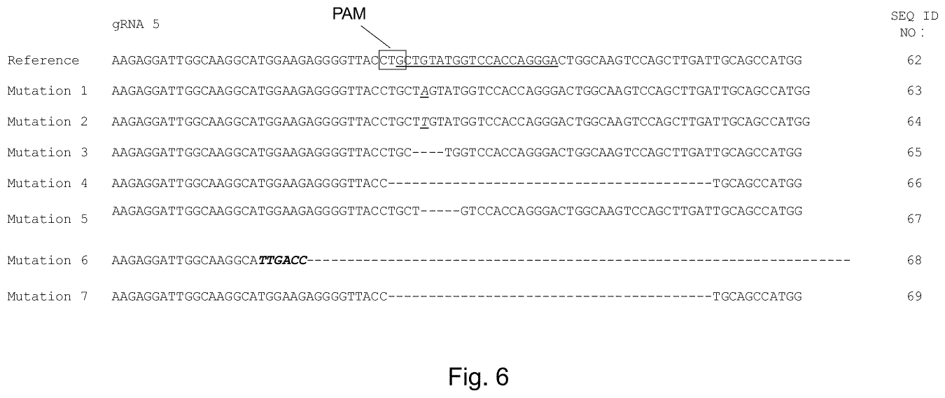

As shown in FIG. 6, there are seven modifications produced at the expect sites in DP2420 rice plants. DP2420H.01A rice plants have the mutant 1 sequence which was shown in SEQ ID NO:45, DP2420H.05A rice plants have the mutant 2 sequence shown in SEQ ID NO:44, and DP2420.06A rice plants have the mutant 3 showed in SEQ ID NO: 46. Mutant 1,2 and 3 resulted early termination of the translation of OsBCS1L coding sequence in the domain of ATPase AAA-type, which further affect the length and the activity of the translated polypeptides.

The genome edited homozygous rice plants were used in the following functional tests.

### Example 5

### Modifying OsBCS1L Gene to Increase Grain Yield in Rice Plants

The OsBCS1L gene over-expressed rice plants (DP0196), OsBCS1L gene suppressed rice plants (DP1200) (described in WO2016/000644), and the OsBCS1L gene edited rice plants (DP2317, DP2354 and DP2420) were planted under well-watered conditions to test the grain yield.

### Method:

About 5 modified rice lines from each gene construct were tested. T2 seeds were sterilized by 800 ppm carbendazol for 8 h at 32° C. and washed 3-5 times with distilled water, then soaked in water for 16 h at 32° C., germinated for 18 h at 35-37° C. in an incubator. The germinated seeds were planted in a seedbed field. At 3-leaf stage, the seedlings were transplanted into the testing field, with 4 replicates and 10 plants per replicate for each transgenic line, and the 4 replicates were planted in the same block. ZH11-TC, DP0158 or the genome edited negative rice plants were nearby the modified lines in the same block and were used as controls in the statistical analysis.

The rice plants were managed by normal practice using pesticides and fertilizers. Watering was normal during the whole growth period.

Plant phenotypes were observed and recorded during the experiments. The phenotypes include heading date, leaf rolling degree, drought sensitivity and drought tolerance. Special attention was paid to leaf rolling degree at noontime. At the end of the growing season, six representative plants of each transgenic line were harvested from the middle of the row per line, and grain weight per plant was measured.

### Results:

1) Grain yield of OsBCS1L genome edited rice plants in the first experiment

The rice plants were planted in the paddy field and the genome edited negative rice plants (with wild-type OsBCS1L gene and no transgenes including Cas9) were used as control. The plants were well watered and no significant difference in phenotype was observed during the full growth period among controls and the mutated plants. The grain yield per plant is shown in Table 3, the grain yield per plant of OsBCS1L over-expressed rice plants(DP0196) were significantly less than the control, the grain yield per plant of OsBCS1L suppressed rice plants were comparable to the control, and the gene edited rice plants showed more enhanced grain yield per plant than the control.

TABLE 3
Grain yield analysis under well-watered conditions at the
construct level (1^{st} experiment)

| Construct ID | Number of Plants | Grain yield per plant (g) | Diff (%) |
|---|---|---|---|
| Control | 24 | 19.17 | |
| DP0196 | 15 | 10.86 | −43 |
| DP1200 | 24 | 18.45 | −4 |
| DP2420 | 64 | 22.01 | 15 |
| DP2317 | 72 | 22.28 | 16 |
| DP2354 | 63 | 21.59 | 13 |

2) Grain yield of OsBCS1L genome edited rice plants in the second experiment

The OsBCS1L genome edited rice plants DP2317, DP2354 and DP2420 were planted under well-water conditions and the grain yield per plant were tested again. In this experiment, totally 200 rice plants from each line were planted with four replicates. DP0158 and the genome edited negative rice plants (with wild-type OsBCS1L gene and no transgenes including Cas9) were used as control. The genome edited negative rice plants were designated as Negative in Table 4, 5 and 6. No significant difference in phenotype was observed during the full growth period among controls and the mutated plants.

As described in Example 4, OsBCS1L gene editing resulted in 14 modifications at the expect sites of DP2317 rice plants, further resulted in translation shift or early termination.

The modifications in the OsBCS1L gene of DP2317P.0113.01, DP2317P.02B.05, DP2317P.0313.01, DP2317P.04B.03 and DP2317P.1013.19 rice plants resulted in translation shift, but the translations were not terminated at the original termination code site. The translated polypeptides may have more amino acid residues than OsBCS1L at the N-terminal end. The detail grain yield results of these modified lines were shown in Table 4. The grain yield per plant of the translation shift lines was equal to that of DP0158 and Negative rice plants at construct level. Only one line showed significantly lower grain yield per plant at line level.

The modifications in the OsBCS1L gene of DP2317P.05B.24, DP2317P.1113.28 and DP2317P.1113.05 rice plants resulted in early termination of the coding sequence and further resulted in 443 to 454 amino acid residues in length. As shown in Table 4, these three lines showed greater grain yield per plant than DP0158 and significantly greater grain yield per plant than the Negative at construct level and two plants showed greater grain yield per plant at line level.

TABLE 4
Grain yield analysis of OsBCS1L gene edited (DP2317) rice plants under well-watered
conditions at the line level (2^{nd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2317F | | 24.06 | 0.25 | 0.922 | | −0.20 | 0.482 | |
| (Construct) | | | | | | | | |
| DP0158 | | 23.80 | | | | | | |
| Negative | | 24.26 | | | | | | |
| DP2317P.0113.01 | 144 | 24.80 | 1.00 | 0.246 | | 0.55 | 0.032 | Y |
| DP2317P.0213.05 | 152 | 24.53 | 0.73 | 0.699 | | 0.27 | 0.885 | |
| DP2317P.0313.01 | 153 | 23.33 | −0.48 | 0.801 | | −0.93 | 0.147 | |
| DP2317P.0413.03 | 143 | 25.28 | 1.48 | 0.597 | | 1.02 | 0.539 | |
| DP2317P.0513.05 | 136 | 25.35 | 1.55 | 0.573 | | 1.09 | 0.729 | |
| DP2317P.1013.19 | 155 | 18.51 | −5.29 | 0.071 | | −5.74 | 0.000 | |
| DP2317T | | 26.11 | 2.30 | 0.379 | | 1.85 | 0.268 | Y |
| (Construct) | | | | | | | | |
| DP0158 | | 23.80 | | | | | | |
| Negative | | 24.26 | | | | | | |
| DP2317P.0513.24 | 131 | 27.52 | 3.72 | 0.139 | | 3.26 | 0.029 | Y |
| DP2317P.1113.28 | 151 | 26.92 | 3.12 | 0.320 | | 2.66 | 0.246 | |
| DP2317P.1113.05 | 134 | 23.62 | −0.18 | 0.971 | | −0.64 | 0.225 | |

As described in example 4, the modifications of OsBCS1L gene resulted in 12 variants at the expect sites in DP2354 rice plants. All these 12 mutants resulted in early termination of translation. The translated polypeptides have 244 to 284 amino acid residues in length. The early termination of the translation of OsBCS1L coding sequence in the domain of ATPase AAA-type, which further affect the length and the activity of the translated polypeptides.

As shown in Table 5, the grain yield per plant of DP2354 rice were equal to that of DP0158 and the Negative rice plants at construct and line level. The difference of grain yield per plant among the DP2354 rice and the DP0158 and Negative were not reach significant level.

TABLE 5
Grain yield analysis of OsBCS1L gene edited (DP2354) rice plants under well-watered
conditions at the line level (2^{nd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2354 | | 24.63 | 0.83 | 0.745 | | 0.37 | 0.368 | |
| (Construct) | | | | | | | | |
| DP0158 | | 23.80 | | | | | | |
| Negative | | 24.26 | | | | | | |
| DP2354P.0113.11 | 153 | 25.37 | 1.57 | 0.605 | | 1.11 | 0.267 | |
| DP2354P.0213.08 | 135 | 25.74 | 1.93 | 0.496 | | 1.48 | 0.206 | |
| DP2354P.0313.10 | 152 | 23.21 | −0.59 | 0.814 | | −1.04 | 0.437 | |
| DP2354P.0413.09 | 155 | 24.17 | 0.37 | 0.902 | | −0.08 | 0.864 | |
| DP2354P.0413.12 | 147 | 23.62 | −0.18 | 0.934 | | −0.64 | 0.967 | |
| DP2354P.0713.07 | 133 | 25.62 | 1.81 | 0.399 | | 1.36 | 0.036 | Y |

As shown in Table 6, the grain yield per plant of DP2420 rice were equal to that of DP0158 and the Negative rice plants at construct level. Six lines showed greater grain yield per plant than both controls at line level.

TABLE 6
Grain yield analysis of OsBCS1L gene edited (DP2420) rice plants under well-watered
conditions at the line level (2^{nd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2420 | | 24.34 | 0.54 | 0.829 | | 0.09 | 0.420 | |
| (Construct) | | | | | | | | |
| DP0158 | | 23.80 | | | | | | |
| Negative | | 24.26 | | | | | | |
| DP2420H.0113.02 | 150 | 20.31 | −3.49 | 0.212 | | −3.95 | 0.106 | |
| DP2420H.0213.08 | 160 | 25.22 | 1.41 | 0.574 | | 0.96 | 0.341 | |
| DP2420H.0313.09 | 151 | 27.03 | 3.23 | 0.323 | | 2.77 | 0.111 | |
| DP2420H.0413.04 | 149 | 24.62 | 0.82 | 0.801 | | 0.36 | 0.484 | |
| DP2420H.0513.02 | 151 | 25.49 | 1.69 | 0.565 | | 1.24 | 0.294 | |
| DP2420H.0613.02 | 159 | 26.79 | 2.99 | 0.320 | | 2.54 | 0.062 | Y |
| DP2420P.0513.04 | 160 | 18.67 | −5.13 | 0.066 | | −5.58 | 0.001 | |
| DP2420P.0813.01 | 151 | 27.05 | 3.24 | 0.237 | | 2.79 | 0.019 | Y |

### Example 6

### Modifying OsBCS1L Gene to Improve Drought Tolerance in Rice Plants

Flowering stage drought stress is an important problem in agriculture practice. The modified rice plants were tested under field drought conditions.

### Method:

9-12 modified lines from each gene construct were tested. T1 and T2 seeds geminated as described in Example 5 and were transplanted into the testing field, with 4 replicates and 10 or 50 plants per replicate for each line, and the 4 replicates were planted in the same block. The OsBCS1L genome edited rice plants (with wild-type OsBCS1L gene and no transgenes including Cas9) and DP0158 rice were in the same block, and were used as controls in the statistical analysis.

The rice plants were managed by normal practice using pesticides and fertilizers. Watering was stopped at the panicle initiation stage, so as to give drought stress at flowering stage depending on the weather conditions (temperature and humidity). The soil water content was measured every 4 days at about 10 sites per block using TDR30 (Spectrum Technologies, Inc.).

At the end of the growing season, representative plants of each transgenic line were harvested from the middle of the row per line, and grain weight per plant was measured. The grain weight data were statistically analyzed using mixed linear model. Positive transgenic lines were selected based on the analysis (P<0.1).

### Results:

### 1) Frist Experiment

The T1 seeds of OsBCS1L gene edited rice plants (DP2420), OsBCS1L over-expressed rice plants (DP0196) and OsBCS1L suppressed rice plants (DP1200) were planted in the same block, and the genome edited negative rice plants which went through the transformation process and have the wild-type (un-mutated) OsBCS1L gene were used as control. Watering was stopped when the main stem panicles were at panicle initiation stage III. The soil volumetric water content decreased slowly from 16% to 6%. 22 days later, the rice plants were at heading stage. The genome edited rice plants did not show drought stress phenotype before dough stage, and showed good setting rate at the maturity stage. As shown in Table 7, the OsBCS1L over-expressed rice plants showed lower grain yield per plant than the control, the OsBCS1L suppressed rice plants and the gene edited rice plants showed more grain yield per plant than the control. These results indicated that reducing the expression of OsBCS1L gene or reducing the activity of OsBCS1L increased the grain yield per plant after drought stress. Further analysis was shown in Table 8, the DP2420 plants obtained greater grain yield per plant than the genome edited negative rice at the line level.

TABLE 7
Grain yield analysis after drought stress at the
construct level (1^{st} experiment)

| | Number of | Grain yield per | | |
|---|---|---|---|---|
| Construct ID | Plants | plant (g) | Diff (%) | P value |
| Control | 180 | 4.63 ± 1.94 | | |
| DP0196 | 78 | 1.90 ± 0.69 | −59 | 0.0002 |
| DP1200 | 123 | 5.73 ± 2.20 | 24 | 0.1062 |
| DP2420 | 112 | 7.12 ± 2.00 | 54 | 0.0003 |

TABLE 8
Grain yield analysis of OsBCS1L gene edited (DP2420) rice plants
after field drought stress at line level (1^{st} experiment)

| | Number | Yield per | | | |
|---|---|---|---|---|---|
| Line ID | of plants | plant (g) | Diff | P value | P ≤ 0.1 |
| Control | 74 | 4.63 | | | |
| DP2420 (construct) | | 7.12 | 2.50 | 0.000 | Y |
| DP2420H.01A | 39 | 8.47 | 3.85 | 0.000 | Y |
| DP2420H.05A | 51 | 6.10 | 1.47 | 0.095 | Y |
| DP2420H.06A | 22 | 6.80 | 2.17 | 0.024 | Y |

### 2) Second Experiment

The T2 seeds of OsBCS1L gene edited rice plants (DP2317, DP2354 and DP2420), OsBCS1L over-expressed rice plants (DP0196) and OsBCS1L suppressed rice plants (DP1200) were planted in the same block in Hainan field, and the genome edited negative rice plants which went through the transformation process and have the wild-type (un-mutated) OsBCS1L gene were used as control. Watering was stopped when the main stem panicles were at panicle initiation stage III. The soil volumetric water content decreased slowly from 35% to 5%. 21 days later, the rice plants were at heading stage, and some rice plants showed leaf rolling phenotype. As shown in table 9, the OsBCS1L over-expressed rice plants showed significantly lower grain yield per plant than the control, the OsBCS1L suppressed rice plants showed more grain yield per plant, and all gene edited rice plants showed more grain yield per plant than the control at the construct level. These results further demonstrate that reducing the expression of OsBCS1L gene increased the grain yield per plant, reducing the activity of OsBCS1L also increased the grain yield per plant after drought stress.

TABLE 9
Grain yield analysis after drought stress at the construct
level (2^{nd} experiment)

| | Number | Grain yield | | |
|---|---|---|---|---|
| Construct ID | of Plants | per plant (g) | Diff (%) | P value |
| Control | 180 | 5.21 ± 1.13 | | |
| DP0196 | 96 | 2.90 ± 1.68 | −44 | 0.0002 |
| DP1200 | 144 | 6.18 ± 2.85 | 19 | 0.0981 |
| DP2317 | 426 | 7.49 ± 2.25 | 44 | 0.0000 |
| DP2354 | 427 | 7.11 ± 2.65 | 36 | 0.0001 |
| DP2420 | 383 | 7.53 ± 2.33 | 45 | 0.0000 |

The modifications in the OsBCS1L gene of DP2317P.0113.01, DP2317P.02B.05, DP2317P.0313.01, DP2317P.04B.03 and DP2317P.1013.19 rice plants resulted in translation shift, but the translations were not terminated at the original termination code site. The translated polypeptides may have more amino acid residues than OsBCS1L at the N-terminal end. The detail grain yield results of these modified lines were shown in Table 10. Four lines showed more grain yield per plant than the control, one lines showed slightly less grain yield per plant than the control at line level.

The modifications in the OsBCS1L gene of DP2317P.05B.24, DP2317P.1113.28 and DP2317P.116.05 rice plants resulted in early termination of the coding sequence and further resulted in 443 to 454 amino acid residues in length. As shown in Table 10, all these three lines showed significantly greater grain yield per plant than the control at line level.

TABLE 10
Grain yield analysis of OsBCS1L gene edited (DP2317) rice plants
after field drought stress at line level

| | Number | Grain yield | | | |
|---|---|---|---|---|---|
| Line ID | of plants | per plant (g) | Diff | P value | P ≤ 0.1 |
| Control | 144 | 5.21 | | | |
| DP2317F | | 5.91 | 0.70 | 0.162 | |
| (construct) | | | | | |
| DP2317P.0113.01 | 48 | 6.49 | 1.28 | 0.099 | Y |
| DP2317P.0213.05 | 42 | 7.01 | 1.79 | 0.020 | Y |
| DP2317P.0313.01 | 48 | 5.56 | 0.35 | 0.648 | |
| DP2317P.0413.03 | 48 | 5.82 | 0.61 | 0.433 | |
| DP2317P.1013.19 | 48 | 4.70 | −0.51 | 0.513 | |
| DP2317T | | 9.00 | 3.79 | 0.000 | Y |
| (construct) | | | | | |
| DP2317P.0513.24 | 48 | 8.64 | 3.43 | 0.000 | Y |
| DP2317P.1113.28 | 48 | 10.89 | 5.68 | 0.000 | Y |
| DP2317P.1113.05 | 48 | 7.48 | 2.27 | 0.004 | Y |

As shown in Table 11, six DP2354 modified lines showed greater grain yield per plant than the control, wherein the three lines which exhibited good seed setting phenotype lines at the maturity stage showed significantly greater grain yield per plant.

TABLE 11
Grain yield analysis of OsBCS1L gene edited (DP2317) rice plants
after field drought stress at line level

| | Number | Grain yield | | | |
|---|---|---|---|---|---|
| Line ID | of plants | per plant (g) | Diff | P value | P ≤ 0.1 |
| Control | 144 | 5.21 | | | |
| DP2354 (construct) | | 7.11 | 1.90 | 0.000 | Y |
| DP2354P.0113.11 | 48 | 6.59 | 1.38 | 0.072 | |
| DP2354P.0213.08 | 48 | 6.37 | 1.16 | 0.127 | |
| DP2354P.0313.10 | 48 | 6.19 | 0.98 | 0.207 | |
| DP2354P.0413.09 | 48 | 9.30 | 4.09 | 0.000 | Y |
| DP2354P.0413.12 | 48 | 9.12 | 3.91 | 0.000 | Y |
| DP2354P.0513.11 | 48 | 4.36 | −0.85 | 0.276 | |
| DP2354P.0713.07 | 47 | 7.86 | 2.65 | 0.001 | Y |

In the second experiment, two modified lines DP2420H.016.02, and DP2420P.0813.01 showed good seed setting phenotype at maturity stage. As shown in Table 12, five of the seven tested lines showed significantly greater grain yield per plant than the control.

TABLE 12
Grain yield analysis of OsBCS1L gene edited (DP2420) rice plants
after field drought stress at line level (2^{nd} experiment)

| | Number | Grain yield | | | |
|---|---|---|---|---|---|
| Line ID | of plants | per plant (g) | Diff | P value | P ≤ 0.1 |
| Control | 144 | 5.21 | | | |
| DP2420 (Construct) | | 7.53 | 2.26 | 0.000 | Y |
| DP2420H.0113.02 | 48 | 8.80 | 3.59 | 0.000 | Y |
| DP2420H.0213.08 | 48 | 7.46 | 2.25 | 0.004 | Y |
| DP2420H.0313.09 | 48 | 7.95 | 2.74 | 0.000 | Y |
| DP2420H.0413.04 | 48 | 5.13 | −0.08 | 0.918 | |
| DP2420H.0513.02 | 48 | 6.88 | 1.67 | 0.033 | Y |
| DP2420H.0613.02 | 48 | 6.60 | 1.39 | 0.107 | |
| DP2420P.0813.01 | 48 | 9.90 | 4.68 | 0.000 | Y |

### 3) Third Experiment

The OsBCS1L gene edited rice plants (DP2317, DP2354 and DP2420) were planted in the same block in Hainan field and tested again under drought conditions. DP0158 rice plants and the genome edited negative rice plants were used as controls. Watering was stopped when the main stem panicles were at panicle initiation stage II. The soil volumetric water content decreased slowly from 35% to 15%. 32 days later, the rice plants were at heading stage, and some rice plants showed leaf rolling phenotype.

As shown in table 13, the OsBCS1L gene edited rice plants (DP2317) in which mutant resulted in translation shift showed similar grain yield per plant to the controls at the construct level. Only one line DP2317P.0113.01 showed significantly greater grain yield per plant at the line level. The OsBCS1L gene edited rice plants (DP2317) with early termination of the coding sequence showed significantly greater grain yield per plant than the controls at the construct level, wherein the two lines which exhibited good seed setting phenotype at the maturity stage showed significantly greater grain yield per plant.

TABLE 13
Grain yield analysis of OsBCS1L gene edited (DP2317) rice plants after field
drought stress at line level (3^{rd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2317F | | 3.93 | 0.20 | 0.625 | | −0.04 | 0.922 | |
| DP0158 | | 3.72 | | | | | | |
| Negative | | 3.96 | | | | | | |
| DP2317P.0113.01 | 156 | 4.77 | 1.05 | 0.021 | Y | 0.80 | 0.063 | Y |
| DP2317P.0213.05 | 146 | 4.17 | 0.45 | 0.488 | | 0.20 | 0.753 | |
| DP2317P.0313.01 | 157 | 3.65 | −0.07 | 0.915 | | −0.31 | 0.637 | |
| DP2317P.0413.03 | 144 | 4.56 | 0.84 | 0.257 | | 0.59 | 0.419 | |
| DP2317P.0513.05 | 154 | 3.33 | −0.39 | 0.570 | | −0.63 | 0.338 | |
| DP2317P.1013.19 | 136 | 3.07 | −0.65 | 0.345 | | −0.90 | 0.174 | |
| DP2317T | | 5.48 | 1.75 | 0.001 | Y | 1.51 | 0.002 | Y |
| DP0158 | | 3.72 | | | | | | |
| Negative | | 3.96 | | | | | | |
| DP2317P.0513.24 | 153 | 4.34 | 0.62 | 0.356 | | 0.38 | 0.559 | |
| DP2317P.1113.28 | 144 | 5.79 | 2.06 | 0.002 | Y | 1.82 | 0.006 | Y |
| DP2317P.1113.05 | 140 | 6.30 | 2.58 | 0.000 | Y | 2.34 | 0.001 | Y |

As shown in Table 14, the OsBCS1L gene edited rice plants (DP2354) showed significantly greater grain yield per plant than the controls at construct level, and five lines showed significantly greater grain yield per plant at line level.

TABLE 14
Grain yield analysis of OsBCS1L gene edited (DP2354) rice plants after field
drought stress at line level (3^{rd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2354 | 906 | 5.21 | 1.49 | 0.001 | Y | 1.25 | 0.002 | Y |
| (Construct) | | | | | | | | |
| DP0158 | | 3.72 | | | | | | |
| Negative | | 3.96 | | | | | | |
| DP2354P.0113.11 | 154 | 5.26 | 1.54 | 0.024 | Y | 1.29 | 0.054 | Y |
| DP2354P.0213.08 | 158 | 4.56 | 0.84 | 0.211 | | 0.60 | 0.366 | |
| DP2354P.0313.10 | 155 | 5.17 | 1.45 | 0.035 | Y | 1.21 | 0.058 | Y |
| DP2354P.0413.09 | 157 | 5.67 | 1.95 | 0.005 | Y | 1.71 | 0.012 | Y |
| DP2354P.0413.12 | 144 | 5.47 | 1.75 | 0.013 | Y | 1.51 | 0.025 | Y |
| DP2354P.0713.07 | 138 | 5.14 | 1.41 | 0.001 | Y | 1.17 | 0.007 | Y |

As shown in Table 15, the OsBCS1L gene edited rice plants (DP2420) showed significantly greater grain yield per plant than the controls at construct level, and three lines showed significantly greater grain yield per plant at line level.

TABLE 15
Grain yield analysis of OsBCS1L gene edited (DP2420) rice plants after field
drought stress at line level (3^{rd} experiment)

| | Number of | Yield per | | | | | | |
|---|---|---|---|---|---|---|---|---|
| | harvested | plant | CK = DP0158 | | | CK = Negative | | |
| Line ID | plants | (g) | Diff | P value | P ≤ 0.1 | Diff | P value | P ≤ 0.1 |
| DP2420 | | 4.83 | 1.11 | 0.007 | Y | 0.87 | 0.033 | Y |
| (Construct) | | | | | | | | |
| DP0158 | | 3.72 | | | | | | |
| Negative | | 3.96 | | | | | | |
| DP2420H.0213.08 | 153 | 4.48 | 0.76 | 0.267 | | 0.51 | 0.448 | |
| DP2420H.0313.09 | 154 | 4.47 | 0.74 | 0.265 | | 0.50 | 0.448 | |
| DP2420H.0413.04 | 154 | 3.39 | −0.33 | 0.610 | | −0.57 | 0.364 | |
| DP2420H.0513.02 | 144 | 6.32 | 2.60 | 0.000 | Y | 2.36 | 0.000 | Y |
| DP2420H.0613.02 | 156 | 5.96 | 2.24 | 0.002 | Y | 2.00 | 0.004 | Y |
| DP2420P.0513.04 | 152 | 3.24 | −0.48 | 0.473 | | −0.72 | 0.283 | |
| DP2420P.0813.01 | 143 | 5.97 | 2.25 | 0.001 | Y | 2.01 | 0.004 | Y |

The results in three experiments demonstrated that reducing the expression of OsBCS1L gene increased the grain yield per plant, reducing the activity of OsBCS1L also increased the grain yield per plant after drought stress.

### Example 7

### Laboratory Paraquat Assays of Modified Rice Plants

Paraquat (1,1-dimethyl-4,4-bipyridinium dichloride), is a foliar-applied and non-selective bipyridinium herbicide, and it is one of the most widely used herbicides in the world, controlling weeds in a huge variety of crops like corn, rice, soybean etc. In plant cells, paraquat mainly targets chloroplasts by accepting electrons from photosystem I and then reacting with oxygen to produce superoxide and hydrogen peroxide, which cause photooxidative stress. Drought stress and cold stress usually leads to increased reactive oxygen species (ROS) in plants and sometimes, the drought and/or cold tolerance of plant is associated with enhanced antioxidative ability. Paraquat is a potent oxidative stress inducer; it greatly increases the ROS production and inhibits the regeneration of reducing equivalents and compounds necessary for the activity of the antioxidant system. The ROS generation is enhanced under abiotic stress conditions, and the plant responses range from tolerance to death depending on the stress intensity and its associated-ROS levels. Relative low level of paraquat can mimic the stress-associated ROS production and used as a stress tolerance marker in plant stress biology (Hasaneen M.N.A. (2012) Herbicide-Properties, Synthesis and Control of Weeds book). Therefore, the paraquat tolerance of genome edited rice plants was tested.

### Paraquat Assay Methods:

OsBCS1L modified rice plants from eight modified lines were tested by paraquat assay. Tissue-cultured Zhonghua 11 plants (ZH11-TC) and empty vector transgenic plants (DP0158) were used as controls. T3 seeds were sterilized and germinated as described in Example 4, and this assay was carried out in growth room with temperature at 28-30 ° C. and humidity ˜30%. The germinated seeds were placed in a tube with a hole at the bottom, and water cultured at 30° C. for 5 days till one-leaf and one-terminal bud stage. Uniform seedlings about 3.5-4 cm in height were selected for paraquat testing. Randomized block design was used in this experiment. There were five blocks, each of which has 16×12 holes. Each modified line was placed in one row (12 plants/line), and ZH11-TC and DP0158 seedlings were placed in 3 rows (3×12 plants) randomly in one block. Then the seedlings were treated with 0.8 μM paraquat solution for 7 days at 10 h day/14 h night, and the treated seedlings first encountered dark and took up the paraquat solution which was changed every two days. After treated for 7 days, the green seedlings were counted. Those seedlings that maintain green in whole without damage were considered as paraquat tolerant seedling; those with bleached leaves or stem were not considered as paraquat tolerant seedling.

Tolerance rate was used as a parameter for this trait screen, which is the percentage of plants which kept green and showed tolerant phenotype over the total plant number.

The data was analyzed at construct level (all modified plants compared with the control) and line level (different modified lines compared with the control) using a statistic model of “Y˜seg+line (seg)+rep+error”, random effect of “rep”, Statistic Method of “SAS® PROC GLIMMIX”.

### Paraquat Assay Results:

1) Paraquat validation results of OsBCS1L gene edited (DP2317) rice plants

In the first experiment, after paraquat solution treated for seven days, 330 of the 480 DP2317 seedlings (69%) kept green and showed tolerant phenotype, while 92 of the 120 (77%) seedlings from ZH11-TC showed tolerant phenotype, and 84 of the 120 (70%) DP0158 seedlings showed tolerant phenotype. The tolerance rate of all screened DP2317 seedlings was less than ZH11-TC and DP0158 controls at the construct level.

Further analysis at line level indicates that the difference of the tolerance rate among the genome edited line and the controls were small and didn't reach significant level (Table 16).

TABLE 16
Paraquat tolerance assay of OsBCS1L gene edited (DP2317) rice plants under
laboratory conditions (1^{st} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2317 (Construct) | 330 | 480 | 69 | 0.1019 | | 0.8175 | |
| ZH11-TC | 92 | 120 | 77 | | | | |
| DP0158 | 84 | 120 | 70 | | | | |
| DP2317P.0113.01 | 35 | 60 | 58 | 0.0143 | | 0.1251 | |
| DP2317P.0113.03 | 43 | 60 | 72 | 0.4680 | | 0.8166 | |
| DP2317P.0213.05 | 41 | 60 | 68 | 0.2356 | | 0.8204 | |
| DP2317P.0313.06 | 40 | 60 | 67 | 0.1589 | | 0.6509 | |
| DP2317P.0413.23 | 43 | 60 | 72 | 0.4680 | | 0.8166 | |
| DP2317P.0513.05 | 48 | 60 | 80 | 0.6139 | | 0.1594 | |
| DP2317P.1113.28 | 38 | 60 | 63 | 0.0406 | | 0.2665 | |
| DP2317P.1613.06 | 42 | 60 | 70 | 0.3375 | | 0.9997 | |

The results in the second experiment showed the similar trend. The tolerance rate of all screened DP2317 seedlings was similar to ZH11-TC and DP0158 controls at the construct level. Only one line showed significantly greater paraquat tolerance rate than ZH11-TC control at line level (Table 17). These results demonstrate that DP2317 rice plants didn't enhance paraquat tolerance compared to both controls of ZH11-TC and DP0158 rice plants at construct and transgenic line level at seedling stages.

TABLE 17
Paraquat tolerance assay of OsBCS1L gene edited (DP2317) rice plants under
laboratory conditions (2^{nd} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2317 | 309 | 480 | 64 | 0.1865 | | 0.6637 | |
| ZH11-TC | 106 | 180 | 59 | | | | |
| DP0158 | 79 | 120 | 66 | | | | |
| DP2317P.0113.01 | 36 | 60 | 60 | 0.8796 | | 0.3823 | |
| DP2317P.0113.03 | 45 | 60 | 75 | 0.0306 | Y | 0.2585 | |
| DP2317P.0213.05 | 40 | 60 | 67 | 0.2901 | | 0.9998 | |
| DP2317P.0313.06 | 39 | 60 | 65 | 0.4054 | | 0.8244 | |
| DP2317P.0413.23 | 38 | 60 | 63 | 0.5450 | | 0.6588 | |
| DP2317P.0513.05 | 37 | 60 | 62 | 0.7052 | | 0.5100 | |
| DP2317P.1113.28 | 36 | 60 | 60 | 0.8796 | | 0.3823 | |
| DP2317P.1613.06 | 38 | 60 | 63 | 0.5450 | | 0.6588 | |

2) Paraquat validation results of OsBCS1L gene edited (DP2354) rice plants

In the first experiment, after paraquat solution treated for seven days, 335 of the 480

DP2354 seedlings (70%) kept green and showed tolerant phenotype, while 94 of the 120 (78%) seedlings from ZH11-TC showed tolerant phenotype, and 85 of the 120 (71%) DP0158 seedlings showed tolerant phenotype. The tolerance rate of all screened DP2354 seedlings was less than ZH11-TC and DP0158 controls at the construct level.

Further analysis at line level indicates that the tolerance rate of DP2354 rice plants less than ZH11-TC control and three lines showed slightly higher tolerance rate than DP0158 control (Table 18).

TABLE 18
Paraquat tolerance assay of OsBCS1L gene edited (DP2354) rice plants under
laboratory conditions (1^{st} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2354 | 335 | 480 | 70 | 0.0681 | | 0.8144 | |
| ZH11-TC | 94 | 120 | 78 | | | | |
| DP0158 | 85 | 120 | 71 | | | | |
| DP2354P.0213.10 | 46 | 60 | 77 | 0.8005 | | 0.4099 | |
| DP2354P.0413.14 | 40 | 60 | 67 | 0.0971 | | 0.5688 | |
| DP2354P.0513.03 | 40 | 60 | 67 | 0.0372 | | 0.3117 | |
| DP2354P.0613.02 | 39 | 60 | 65 | 0.0609 | | 0.4281 | |
| DP2354P.0713.03 | 47 | 60 | 78 | 0.9997 | | 0.2879 | |
| DP2354P.0713.05 | 37 | 60 | 62 | 0.0221 | | 0.2196 | |
| DP2354P.0813.05 | 41 | 60 | 68 | 0.1503 | | 0.7309 | |
| DP2354P.0813.14 | 45 | 60 | 75 | 0.6146 | | 0.5596 | |

The results in the second experiment showed the similar trend. The tolerance rate of all screened DP2354 seedlings was similar to ZH11-TC and DP0158 controls at the construct and line level (Table 19). These results demonstrate that DP2354 rice plants didn't enhance paraquat tolerance compared to both controls of ZH11-TC and DP0158 rice plants at construct and transgenic line level at seedling stages.

TABLE 19
Paraquat tolerance assay of OsBCS1L gene edited (DP2354) rice plants
under laboratory conditions (2^{nd} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2354 | 237 | 480 | 49 | 0.7827 | | 0.4948 | |
| ZH11-TC | 91 | 180 | 51 | | | | |
| DP0158 | 55 | 120 | 46 | | | | |
| DP2354P.0213.10 | 31 | 60 | 52 | 0.8820 | | 0.4632 | |
| DP2354P.0413.14 | 29 | 60 | 48 | 0.7666 | | 0.7524 | |
| DP2354P.0513.03 | 23 | 60 | 38 | 0.1071 | | 0.3427 | |
| DP2354P.0613.02 | 33 | 60 | 55 | 0.5531 | | 0.2513 | |
| DP2354P.0713.03 | 31 | 60 | 52 | 0.8820 | | 0.4632 | |
| DP2354P.0713.05 | 25 | 60 | 42 | 0.2383 | | 0.5979 | |
| DP2354P.0813.05 | 32 | 60 | 53 | 0.7106 | | 0.3467 | |
| DP2354P.0813.14 | 33 | 60 | 55 | 0.5531 | | 0.2513 | |

3) Paraquat validation results of OsBCS1L gene edited (DP2420) rice plants

In the first experiment, after paraquat solution treated for seven days, 329 of the 480 DP2420 seedlings (69%) kept green and showed tolerant phenotype, while 81 of the 120 (68%) seedlings from ZH11-TC showed tolerant phenotype, and 86 of the 120 (72%) DP0158 seedlings showed tolerant phenotype. The tolerance rate of all screened DP2420 seedlings was similar to ZH11-TC and DP0158 controls at the construct level.

Further analysis at line level indicates that the difference of the tolerance rate among the genome edited line (DP2420) and the controls were small and didn't reach significant level (Table 20).

TABLE 20
Paraquat tolerance assay of OsBCS1L gene edited (DP2420) rice plants under
laboratory conditions (2^{nd} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2420 | 329 | 480 | 69 | 0.9512 | | 0.5212 | |
| ZH11-TC | 81 | 120 | 68 | | | | |
| DP0158 | 86 | 120 | 72 | | | | |
| DP2420H.0113.05 | 40 | 60 | 67 | 0.8223 | | 0.4931 | |
| DP2420H.0313.02 | 44 | 60 | 73 | 0.4932 | | 0.8148 | |
| DP2420H.0313.09 | 39 | 60 | 65 | 0.6550 | | 0.3643 | |
| DP2420H.0613.02 | 41 | 60 | 68 | 1.0000 | | 0.6454 | |
| DP2420P.0413.04 | 43 | 60 | 72 | 0.6489 | | 1.0000 | |
| DP2420H.0513.01 | 39 | 60 | 65 | 0.6550 | | 0.3643 | |
| DP2420P.0813.01 | 40 | 60 | 67 | 0.8223 | | 0.4931 | |
| DP2420P.0813.02 | 43 | 60 | 72 | 0.6489 | | 1.0000 | |

The results in the second experiment showed the similar trend. The tolerance rate of all screened DP2420 seedlings was lower than ZH11-TC and DP0158 controls at the construct and line level (Table 21). These results demonstrate that DP2420 rice plants didn't enhance paraquat tolerance compared to both controls of ZH11-TC and DP0158 rice plants at construct and transgenic line level at seedling stages.

TABLE 21
Paraquat tolerance assay of OsBCS1L gene edited (DP2420) rice plants under
laboratory conditions (2^{nd} experiment)

| | Number | Number | | CK = ZH11-TC | | CK = DP0158 | |
|---|---|---|---|---|---|---|---|
| | of tolerant | of total | Tolerance | P | | P | |
| Line ID | seedlings | seedlings | rate (%) | value | P ≤ 0.05 | value | P ≤ 0.05 |
| DP2420 | 218 | 420 | 52 | 0.4498 | | 0.3622 | |
| ZH11-TC | 132 | 240 | 55 | | | | |
| DP0158 | 70 | 120 | 58 | | | | |
| DP2420H.0113.05 | 22 | 60 | 37 | 0.0145 | | 0.0146 | |
| DP2420H.0313.02 | 33 | 60 | 55 | 0.9996 | | 0.8323 | |
| DP2420H.0313.09 | 26 | 60 | 43 | 0.1117 | | 0.0971 | |
| DP2420H.0613.02 | 31 | 60 | 52 | 0.6442 | | 0.5267 | |
| DP2420H.0513.01 | 33 | 60 | 55 | 0.9996 | | 0.8323 | |
| DP2420P.0813.01 | 35 | 60 | 58 | 0.6427 | | 0.8315 | |
| DP2420P.0813.02 | 38 | 60 | 63 | 0.2490 | | 0.3950 | |

1. A modified plant or seed comprising an introduced genetic modification in an endogenous BCS1 L gene that decreases the expression or the activity of an endogenous BCS1 L polypeptide, when compared to the expression or the activity of the wild-type BCS1 L polypeptide in a control plant, wherein the plant exhibits at least one phenotype selected from the group consisting of: increased grain yield, increased abiotic stress tolerance and increased biomass, compared to the control plant.

2. The modified plant or seed of claim 1, wherein the introduced genetic modification comprises introducing a DNA fragment, deleting a DNA fragment, replacing a DNA fragment or introducing one or more nucleotide changes in the genomic region comprising the endogenous BCS1L gene and its promoter.

3. The modified plant or seed of claim 2, wherein the introduced genetic modification results in early termination of the coding sequence of the BCS1L gene thereby resulting in a shortened BCS1L polypeptide.

4. (canceled)

5. The modified plant or seed of claim 1, wherein the introduced genetic modification is in the coding sequence of the BCS1L gene.

6. (canceled)

7. The modified plant or seed of claim 1, wherein the BCS1L gene comprises a nucleotide sequence of at least 85% sequence identity to SEQ ID NO: 6 or 7.

8. The modified plant or seed of claim 1, wherein the introduced genetic modification is in a regulatory element of the endogenous BCS1L gene.

9. The modified plant or seed of claim 8, wherein the regulatory element is the endogenous BCS1L promoter comprising a nucleotide sequence of at least 90% sequence identity to SEQ ID NO: 9.

10. The modified plant or seed of claim 1, wherein the plant exhibits an increase drought tolerance.

11. The modified plant or seed of claim 1, herein said plant is selected from the group consisting of rice, maize, soybean, sunflower, sorghum, canola, wheat, alfalfa, cotton, barley, millet, sugar cane and switchgrass.

12-14. (canceled)

15. A method of increasing drought tolerance in a plant, comprising:
(a) introducing into a regenerable plant cell a genetic modification at a genomic region encoding a BCS1L polypeptide, thereby decreasing the expression or activity of the BCS1L polypeptide; and
(b) regenerating a modified plant from the regenerable plant cell after step (a), wherein the modified plant comprises the introduced genetic modification and exhibits increased drought tolerance when compared to a control plant.

16-18. (canceled)

19. The method of claim 15, wherein the genetic modification is introduced into a regulatory element of the gene.

20. The method of claim 15, wherein the genetic modification is introduced into the coding sequence of the gene.

21. The method of claim 15, wherein the genetic modification is introduced using a zinc finger nuclease, a Transcription Activator-Like Effector Nuclease (TALEN), CRISPR-Cas/Cpf1, or a meganuclease.

22. The method of claim 15, wherein the plant is selected from the group consisting of rice, maize, soybean, sunflower, sorghum, canola, wheat, alfalfa, cotton, barley, millet, sugar cane and switchgrass.

23. The method of claim 15, wherein the method further comprises (c) obtaining a progeny plant from the modified plant of step (b), wherein said progeny plant comprises the genetic modification and exhibits increased drought tolerance when compared to a control plant.


SEQUENCE LISTING (69 sequences)

SEQ ID NO: 1 - DNA, 1934 bp, Zea mays
cagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctcttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgct

SEQ ID NO: 2 - DNA, 4206 bp, Artificial Sequence
  Nucleotide sequence of nuclear localization sequence and Cas9 gene
atggcccctaagaagaagagaaaggtcggtattcacggcgttcctgcggcgatggacaagaagtatagtattggtctggacattgggacgaattccgttggctgggccgtgatcaccgatgagtacaaggtcccttccaagaagtttaaggttctggggaacaccgatcggcacagcatcaagaagaatctcattggagccctcctgttcgactcaggcgagaccgccgaagcaacaaggctcaagagaaccgcaaggagacggtatacaagaaggaagaataggatctgctacctgcaggagattttcagcaacgaaatggcgaaggtggacgattcgttctttcatagattggaggagagtttcctcgtcgaggaagataagaagcacgagaggcatcctatctttggcaacattgtcgacgaggttgcctatcacgaaaagtaccccacaatctatcatctgcggaagaagcttgtggactcgactgataaggcggaccttagattgatctacctcgctctggcacacatgattaagttcaggggccattttctgatcgagggggatcttaacccggacaatagcgatgtggacaagttgttcatccagctcgtccaaacctacaatcagctctttgaggaaaacccaattaatgcttcaggcgtcgacgccaaggcgatcctgtctgcacgcctttcaaagtctcgccggcttgagaacttgatcgctcaactcccgggcgaaaagaagaacggcttgttcgggaatctcattgcactttcgttggggctcacaccaaacttcaagagtaattttgatctcgctgaggacgcaaagctgcagctttccaaggacacttatgacgatgacctggataaccttttggcccaaatcggcgatcagtacgcggacttgttcctcgccgcgaagaatttgtcggacgcgatcctcctgagtgatattctccgcgtgaacaccgagattacaaaggccccgctctcggcgagtatgatcaagcgctatgacgagcaccatcaggatctgacccttttgaaggctttggtccggcagcaactcccagagaagtacaaggaaatcttctttgatcaatccaagaacggctacgctggttatattgacggcggggcatcgcaggaggaattctacaagtttatcaagccaattctggagaagatggatggcacagaggaactcctggtgaagctcaatagggaggaccttttgcggaagcaaagaactttcgataacggcagcatccctcaccagattcatctcggggagctgcacgccatcctgagaaggcaggaagacttctacccctttcttaaggataaccgggagaagatcgaaaagattctgacgttcagaattccgtactatgtcggaccactcgcccggggtaattccagatttgcgtggatgaccagaaagagcgaggaaaccatcacaccttggaacttcgaggaagtggtcgataagggcgcttccgcacagagcttcattgagcgcatgacaaattttgacaagaacctgcctaatgagaaggtccttcccaagcattccctcctgtacgagtatttcactgtttataacgaactcacgaaggtgaagtatgtgaccgagggaatgcgcaagcccgccttcctgagcggcgagcaaaagaaggcgatcgtggaccttttgtttaagaccaatcggaaggtcacagttaagcagctcaaggaggactacttcaagaagattgaatgcttcgattccgttgagatcagcggcgtggaagacaggtttaacgcgtcactggggacttaccacgatctcctgaagatcattaaggataaggacttcttggacaacgaggaaaatgaggatatcctcgaagacattgtcctgactcttacgttgtttgaggatagggaaatgatcgaggaacgcttgaagacgtatgcccatctcttcgatgacaaggttatgaagcagctcaagagaagaagatacaccggatggggaaggctgtcccgcaagcttatcaatggcattagagacaagcaatcagggaagacaatccttgactttttgaagtctgatggcttcgcgaacaggaattttatgcagctgattcacgatgactcacttactttcaaggaggatatccagaaggctcaagtgtcgggacaaggtgacagtctgcacgagcatatcgccaaccttgcgggatctcctgcaatcaagaagggtattctgcagacagtcaaggttgtggatgagcttgtgaaggtcatgggacggcataagcccgagaacatcgttattgagatggccagagaaaatcagaccacacaaaagggtcagaagaactcgagggagcgcatgaagcgcatcgaggaaggcattaaggagctggggagtcagatccttaaggagcacccggtggaaaacacgcagttgcaaaatgagaagctctatctgtactatctgcaaaatggcagggatatgtatgtggaccaggagttggatattaaccgcctctcggattacgacgtcgatcatatcgttcctcagtccttccttaaggatgacagcattgacaataaggttctcaccaggtccgacaagaaccgcgggaagtccgataatgtgcccagcgaggaagtcgttaagaagatgaagaactactggaggcaacttttgaatgccaagttgatcacacagaggaagtttgataacctcactaaggccgagcgcggaggtctcagcgaactggacaaggcgggcttcattaagcggcaactggttgagactagacagatcacgaagcacgtggcgcagattctcgattcacgcatgaacacgaagtacgatgagaatgacaagctgatccgggaagtgaaggtcatcaccttgaagtcaaagctcgtttctgacttcaggaaggatttccaattttataaggtgcgcgagatcaacaattatcaccatgctcatgacgcatacctcaacgctgtggtcggaacagcattgattaagaagtacccgaagctcgagtccgaattcgtgtacggtgactataaggtttacgatgtgcgcaagatgatcgccaagtcagagcaggaaattggcaaggccactgcgaagtatttcttttactctaacattatgaatttctttaagactgagatcacgctggctaatggcgaaatccggaagagaccacttattgagaccaacggcgagacaggggaaatcgtgtgggacaaggggagggatttcgccacagtccgcaaggttctctctatgcctcaagtgaatattgtcaagaagactgaagtccagacgggcgggttctcaaaggaatctattctgcccaagcggaactcggataagcttatcgccagaaagaaggactgggacccgaagaagtatggaggtttcgactcaccaacggtggcttactctgtcctggttgtggcaaaggtggagaagggaaagtcaaagaagctcaagtctgtcaaggagctcctgggtatcaccattatggagaggtccagcttcgaaaagaatccgatcgattttctcgaggcgaagggatataaggaagtgaagaaggacctgatcattaagcttccaaagtacagtcttttcgagttggaaaacggcaggaagcgcatgttggcttccgcaggagagctccagaagggtaacgagcttgctttgccgtccaagtatgtgaacttcctctatctggcatcccactacgagaagctcaagggcagcccagaggataacgaacagaagcaactgtttgtggagcaacacaagcattatcttgacgagatcattgaacagatttcggagttcagtaagcgcgtcatcctcgccgacgcgaatttggataaggttctctcagcctacaacaagcaccgggacaagcctatcagagagcaggcggaaaatatcattcatctcttcaccctgacaaaccttggggctcccgctgcattcaagtattttgacactacgattgatcggaagagatacacttctacgaaggaggtgctggatgcaacccttatccaccaatcgattactggcctctacgagacgcggatcgacttgagtcagctcgggggggataagagaccagcggcaaccaagaaggcaggacaagcgaagaagaagaagtag

SEQ ID NO: 3 - DNA, 367 bp, Cauliflower mosaic virus
cggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggtt

SEQ ID NO: 4 - DNA, 742 bp, Oryza sativa
ctcattagcggtatgcatgttggtagaagtcggagatgtaaataattttcattatataaaaaaggtacttcgagaaaaataaatgcatacgaattaattctttttatgttttttaaaccaagtatatagaatttattgatggttaaaatttcaaaaatatgacgagagaaaggttaaacgtacggcatatacttctgaacagagagggaatatggggtttttgttgctcccaacaattcttaagcacgtaaaggaaaaaagcacattatccacattgtacttccagagatatgtacagcattacgtaggtacgttttctttttcttcccggagagatgatacaataatcatgtaaacccagaatttaaaaaatattctttactataaaaattttaattagggaacgtattattttttacatgacaccttttgagaaagagggacttgtaatatgggacaaatgaacaatttctaagaaatgggcatatgactctcagtacaatggaccaaattccctccagtcggcccagcaatacaaagggaaagaaatgagggggcccacaggccacggcccacttttctccgtggtggggagatccagctagaggtccggcccacaagtggcccttgccccgtgggacggtgggattgcagagcgcgtgggcggaaacaacagtttagtaccacctcgctcacgcaacgacgcgaccacttgcttataagctgctgcgctgaggctcag

SEQ ID NO: 5 - DNA, 83 bp, Artificial Sequence
  Nucleotide sequence of gRNA scaffold
gttttagagctagaaatagcaagttaaaataaggctagtccgttatcaacttgaaaaagtggcaccgagtcggtgcttttttt

SEQ ID NO: 6 - DNA, 2031 bp, Oryza sativa
ccaacttctccttccctcccccccactcctcaccctctcattcaacaccaccccaccgtttcataccatttcaacaccaactctccttccccccccccccccatcccaaaaaaaaaaaaaaaaaacactcctcaccctccccattcaacactactgtttcataccattaccaacaacaaagaggaagagaagttcatcaaaagaagaacaagagaggagccagagcttgctcaccatggcgtcctacgacaaggccatcgagtcatacaagaaggccatcacaaccgctgcatccgttgcagcgtctgtgatgctggtccgcagcgtcgtgaacgagctggttccatacgaggtgcgtgatgtgctgttttccggcctcggctacctgcgttcacaaatttcatctcagcacacaatcatcatcgaggagactgagggctggtcccacaaccacgtctacaacgcggtgcgggcttaccttgcaacacgcatcaacaacaacatgcagcgcctgcgagtcagcagcatggatgaatcttccgagaagatggttgtcaccatggaggaaggtgaagagctggttgatatgcatgagggaacagaattcaaatggtgcttaatctcacgtagcatttcagctgaccccaacaatggcaatggcagcggccaacgtgaggtccgctcctatgagctgagcttccacaggaagcacaaggagaaagccctgaaatcatacctcccattcatcattgctacagccaaggccataaaagaccaggaaagaattctccagatatacatgaatgaatactcagactcatggtctccaattgatctccaccacccatccacattcgacacgcttgccatggaccagaagctgaaacagtcaattattgacgaccttgataggttcatcaagagaaaagattactacaagaggattggcaaggcatggaagaggggttacctgctgtatggtccaccagggactggcaagtccagcttgattgcagccatggcgaatcatctcaagtttgacatatatgatcttgagctgactggggtccattccaactcggagctcagaaggcttctagtcggaatgaccagccggtccattcttgttgttgaggacattgactgtagcatcgaactgaaacaacgggaggcaggggaggaacgtaccaagtccaactctacagaagaagacaagggagaagacaaagtgagtacctttaaaatcagaatgcataataacattcatatttgcaaacctttcatctccttgatttcacgattattctcacctatgctgataagaatttgattgccgtccacacaggtaacattatccgggctgctcaattttgttgatgggctgtggtcaacaagtggagaggaaaggatcatcgttttcacgaccaattacaaggagcgtcttgatcaagcacttatgcggcctggcaggatggacatgcacatccacatggggtactgcaccccagaggctttccggattcttgccagcaactaccactcgatcgactatcatgtcacatatccagagatcgaggagctgatcaaggaggtgatggtgacgcctgcggaggtcgctgaggctctcatgagaaatgatgatattgatgttgcactccttggtctactggagctcctaaagtcaaagataaaagatgccagcgagaccaaggctgaaagcaaggatgcaaataagcagacggaggagaataaagatagcaaagcgatggagaacaaaaatgactcctcaactgatgaatgcacttaggattgtggagtacaacaatgacaacaagaatgtgtatatgctagataggtccaacggaagatccatttcaaaacatgtgatcgttttttgactactaattacaatgtgtatttggagataggaggtgcactggtatttaatgaagtgtgcttcctaagaaatttaaagtattgtctcgacaagtcatacatatgcaacatcagattttgtttgtgtacgaaaca

SEQ ID NO: 7 - DNA, 1455 bp, Oryza sativa
atggcgtcctacgacaaggccatcgagtcatacaagaaggccatcacaaccgctgcatccgttgcagcgtctgtgatgctggtccgcagcgtcgtgaacgagctggttccatacgaggtgcgtgatgtgctgttttccggcctcggctacctgcgttcacaaatttcatctcagcacacaatcatcatcgaggagactgagggctggtcccacaaccacgtctacaacgcggtgcgggcttaccttgcaacacgcatcaacaacaacatgcagcgcctgcgagtcagcagcatggatgaatcttccgagaagatggttgtcaccatggaggaaggtgaagagctggttgatatgcatgagggaacagaattcaaatggtgcttaatctcacgtagcatttcagctgaccccaacaatggcaatggcagcggccaacgtgaggtccgctcctatgagctgagcttccacaggaagcacaaggagaaagccctgaaatcatacctcccattcatcattgctacagccaaggccataaaagaccaggaaagaattctccagatatacatgaatgaatactcagactcatggtctccaattgatctccaccacccatccacattcgacacgcttgccatggaccagaagctgaaacagtcaattattgacgaccttgataggttcatcaagagaaaagattactacaagaggattggcaaggcatggaagaggggttacctgctgtatggtccaccagggactggcaagtccagcttgattgcagccatggcgaatcatctcaagtttgacatatatgatcttgagctgactggggtccattccaactcggagctcagaaggcttctagtcggaatgaccagccggtccattcttgttgttgaggacattgactgtagcatcgaactgaaacaacgggaggcaggggaggaacgtaccaagtccaactctacagaagaagacaagggagaagacaaagtaacattatccgggctgctcaattttgttgatgggctgtggtcaacaagtggagaggaaaggatcatcgttttcacgaccaattacaaggagcgtcttgatcaagcacttatgcggcctggcaggatggacatgcacatccacatggggtactgcaccccagaggctttccggattcttgccagcaactaccactcgatcgactatcatgtcacatatccagagatcgaggagctgatcaaggaggtgatggtgacgcctgcggaggtcgctgaggctctcatgagaaatgatgatattgatgttgcactccttggtctactggagctcctaaagtcaaagataaaagatgccagcgagaccaaggctgaaagcaaggatgcaaataagcagacggaggagaataaagatagcaaagcgatggagaacaaaaatgactcctcaactgatgaatgcacttag

SEQ ID NO: 8 - protein, 484 aa, Oryza sativa
MASYDKAIESYKKAITTAASVAASVMLVRSVVNELVPYEVRDVLFSGLGYLRSQISSQHTIIIEETEGWSHNHVYNAVRAYLATRINNNMQRLRVSSMDESSEKMVVTMEEGEELVDMHEGTEFKWCLISRSISADPNNGNGSGQREVRSYELSFHRKHKEKALKSYLPFIIATAKAIKDQERILQIYMNEYSDSWSPIDLHHPSTFDTLAMDQKLKQSIIDDLDRFIKRKDYYKRIGKAWKRGYLLYGPPGTGKSSLIAAMANHLKFDIYDLELTGVHSNSELRRLLVGMTSRSILVVEDIDCSIELKQREAGEERTKSNSTEEDKGEDKVTLSGLLNFVDGLWSTSGEERIIVFTTNYKERLDQALMRPGRMDMHIHMGYCTPEAFRILASNYHSIDYHVTYPEIEELIKEVMVTPAEVAEALMRNDDIDVALLGLLELLKSKIKDASETKAESKDANKQTEENKDSKAMENKNDSSTDECT

SEQ ID NO: 9 - DNA, 2000 bp, Oryza sativa
aatacaatcacgcacgcacatttacttttatgaacacatactcacgctctactcctgtgtctttggaagaccgtattaacatatcttaagattcacaaagtcgaaacatctttttttttgtcattttccctcgcgaaatctcttttagataatcacactgcgtcaagccaaagatgcactctgcaagtaaggttaatcataaaatattttcatccttaaatatggcactgcaatttgttagggagaaataggaatggtcattttacgcgattgaaacaagtaccggatcatccaagcgattaccacacgtggaccgcataggtgcacacgtgtcagtgactcagtcttgatgatgaagaaatcagaaaccaacgtgccagccacacatgctcaaaaaatgttttttaggaaaaaaataaaaggaaaataaaaaaaacgtgcatgctagattctgctctttctgtcgatggcgccgctaccacccaaagggaaaagggaaaatgttgtttattttgtcttgacactttagataattaattgagttttcctttatggctgctttaaattgcaacacgcagtcccattcacgtttcgatctctcctgcaaaagcaaattaaacgtggcgaccacgattgctttagcaggttcagtcacacgttgtgcccccgaaaacaacacaacacagtgttttgatcatggcagcacagtgtcgacccttgtattcaagcgcgccgcatgatgatcagtaaaatacagttagcgcaacgatctgaaagcaatcaatttgtttgtgatgtgcaacgtgcacgttgctggacgatccaatcgatgccttggcaataggccctcttggagtatcaacgaatcaagagctgcttatggccataaccccaagagctgagtgcacgaaaaacttgtgtgcccagaatacatttacagctcacaaaagtttttaatagtttcatgaggaatctgtatgaacttaaatgtactagataatttccccatcacaacagaagagcacaagagaacagagagaggcggttacacaaggatagaatacgagtagctgtcaccaacagaaacgaataaaagcacccactcaactcactgatgatatcaccaaggaaaatggtccatggagtcgccggacaaaccacagagcaacacgtcgattggtgccaccgccgctacgcgatcaacaatttgacccctttggtccaaaatccatcaccagctgtagcagcagcagcagttgtaaccatggggggctcggttccgtcgatctcaacacgtcggcgacgtagaccgagacaaggcaaagccaaagcaacgccccccactttccccaaatggaccattatatgcatgcagctggaacttttcagaattttataaagttgctgatcgacagtctctgactcggacgtcgaccaaccgtcgtctgcacggaacggtatgcgatttgcgcacatggaatggattcagccaacgaggtggtagatggatgcgggcattcttttctggaatactgatgcgggcattcttctgctccagtatttcgagcagaaacttgcggtgcacaactcttctgctccctgcagaaagaatggttcagaggaaaacttggatgccaaggaagtaaagtttgccttttctagctgcccaaggagtatttggtttattgagatcttaattcgtgacagttaaaacatcattattcaaagttctcttcagtaaagaagatacgctgggaggaataaaggtgcaatagattaagaacaaatgcctcagaaatcgacaatttgtaccggttgcattctttcccaggtgaatatgtgtaggagtattacgttgtaatggtgatgtcattttcctaactgtggaagctggaagtaggagtagtattcagatttctttctttcagacgaagccaccattcgcccccaactctgcacctataaaaccccctccccacaaactctacctcaaatcaaca

SEQ ID NO: 10 - DNA, 20 bp, Artificial Sequence
  Nucleotide sequence of gRNA2 for target site sequence for OsBCS1L gene
gaacgagctggttccatacg

SEQ ID NO: 11 - DNA, 20 bp, Artificial Sequence
  Nucleotide sequence of gRNA3 for target site sequence for OsBCS1L gene
gatcaagcacttatgcggcc

SEQ ID NO: 12 - DNA, 20 bp, Artificial Sequence
  Nucleotide sequence of gRNA4 for target site sequence for OsBCS1L gene
tagcatcgaactgaaacaac

SEQ ID NO: 13 - DNA, 20 bp, Artificial Sequence
  Nucleotide sequence of gRNA5 for target site sequence for OsBCS1L gene
tccctggtggaccatacagc

SEQ ID NO: 14 - DNA, 20 bp, Artificial Sequence
  Nucleotide sequence of gRNA6 for target site sequence for OsBCS1L gene
aaaagatgccagcgagacca

SEQ ID NO: 15 - DNA, 20 bp, Artificial Sequence
  Nucleotide sequence of gRNA8 for target site sequence for OsBCS1L promoter
ttcagtaaagaagatacgct

SEQ ID NO: 16 - DNA, 20 bp, Artificial Sequence
  Nucleotide sequence of gRNA9 for target site sequence for OsBCS1L gene
ccacaaccacgtctacaacg

SEQ ID NO: 17 - DNA, 20 bp, Artificial Sequence
  Nucleotide sequence of gRNA10 for target site sequence for OsBCS1L promoter
gatcaacaatttgacccctt

SEQ ID NO: 18 - DNA, 20 bp, Artificial Sequence
  Nucleotide sequence of gRNA11 for target site sequence for OsBCS1L gene
cataggagcggacctcacgt

SEQ ID NO: 19 - DNA, 20 bp, Artificial Sequence
  Nucleotide sequence of gRNA12 for target site sequence for OsBCS1L gene
ggccttcttgtatgactcga

SEQ ID NO: 20 - DNA, 20 bp, Artificial Sequence
  Nucleotide sequence of gRNA20 for target site sequence for OsBCS1L gene
tgagaataatcgtgaaatca

SEQ ID NO: 21 - DNA, 20 bp, Artificial Sequence
  Nucleotide sequence of gRNA24 for target site sequence for OsBCS1L promoter
tacgcgattgaaacaagtac

SEQ ID NO: 22 - DNA, 20 bp, Artificial Sequence
  Nucleotide sequence of gRNA25 for target site sequence for OsBCS1L promoter
aacagtagtgttgaatgggg

SEQ ID NO: 23 - DNA, 20 bp, Artificial Sequence
  Nucleotide sequence of gRNA26 for target site sequence for OsBCS1L promoter
atggcgccgctaccacccaa

SEQ ID NO: 24 - DNA, 20 bp, Artificial Sequence
  Nucleotide sequence of gRNA27 for target site sequence for OsBCS1L promoter
tccaagagggcctattgcca

SEQ ID NO: 25 - DNA, 20 bp, Artificial Sequence
  Nucleotide sequence of gRNA28 for target site sequence for OsBCS1L promoter
atgcggcgcgcttgaataca

SEQ ID NO: 26 - DNA, 20 bp, Artificial Sequence
  Nucleotide sequence of gRNA29 for target site sequence for OsBCS1L promoter
ataccgttccgtgcagacga

SEQ ID NO: 27 - DNA, 20 bp, Artificial Sequence
  Nucleotide sequence of gRNA30 for target site sequence for OsBCS1L promoter
tgatgtcattttcctaactg

SEQ ID NO: 28 - DNA, 20 bp, Artificial Sequence
  Nucleotide sequence of gRNA31 for target site sequence for OsBCS1L promoter
cagagagaggcggttacaca

SEQ ID NO: 29 - DNA, 20 bp, Artificial Sequence
  Nucleotide sequence of gRNA32 for target site sequence for OsBCS1L gene
tttggagataggaggtgcac

SEQ ID NO: 30 - DNA, 20 bp, Artificial Sequence
  Nucleotide sequence of gRNA33 for target site sequence for OsBCS1L gene
tatatgctagataggtccaa

SEQ ID NO: 31 - DNA, 85 bp, Oryza sativa
ttggtctactggagctcctaaagtcaaagataaaagatgccagcgagaccaaggctgaaagcaaggatgcaaataagcagacgga

SEQ ID NO: 32 - DNA, 75 bp, Oryza sativa
ttggtctactggagctcctaaagtcaaagataaaagatgccagcgagaaagcaaggatgcaaataagcagacgga

SEQ ID NO: 33 - DNA, 84 bp, Oryza sativa
ttggtctactggagctcctaaagtcaaagataaaagatgccagcgacccaaggctgaaagcaaggatgcaaataagcagacgga

SEQ ID NO: 34 - DNA, 81 bp, Oryza sativa
ttggtctactggagctcctaaagtcaaagataaaagatgccagcccaaggctgaaagcaaggatgcaaataagcagacgga

SEQ ID NO: 35 - DNA, 69 bp, Oryza sativa
ttggtctactggagctcctaaagtcaaagataccaaggctgaaagcaaggatgcaaataagcagacgga

SEQ ID NO: 36 - DNA, 11 bp, Oryza sativa
aagcagacgga

SEQ ID NO: 37 - DNA, 84 bp, Oryza sativa
ttggtctactggagctcctaaagtcaaagataaaagatgccagcgagccaaggctgaaagcaaggatgcaaataagcagacgga

SEQ ID NO: 38 - DNA, 53 bp, Oryza sativa
ttggtctactggagctccaaggctgaaagcaaggatgcaaataagcagacgga

SEQ ID NO: 39 - DNA, 86 bp, Oryza sativa
ttggtctactggagctcctaaagtcaaagataaaagatgccagcgagaaccaaggctgaaagcaaggatgcaaataagcagacgga

SEQ ID NO: 40 - DNA, 80 bp, Oryza sativa
ttggtctactggagctcctaaagtcaaagataaaagatgccagcgaataaagatagcaaggatgcaaataagcagacgga

SEQ ID NO: 41 - DNA, 83 bp, Oryza sativa
ttggtctactggagctcctaaagtcaaagataaaagatgccagcgaccaaggctgaaagcaaggatgcaaataagcagacgga

SEQ ID NO: 42 - DNA, 47 bp, Oryza sativa
ttggtctactggagctcctaaagcaaggatgcaaataagcagacgga

SEQ ID NO: 43 - DNA, 76 bp, Oryza sativa
ttggtctactggagctcctaaagtcaaagataaaagatgccaaggctgaaagcaaggatgcaaataagcagacgga

SEQ ID NO: 44 - DNA, 85 bp, Oryza sativa
ttggtctactggagctcctaaagtcaaagataaaagatgccagcgagaatgcaaatgaaagcaaggatgcaaataagcagacgga

SEQ ID NO: 45 - DNA, 49 bp, Oryza sativa
atgccagcgagaccaaggctgaaagcaaggatgcaaataagcagacgga

SEQ ID NO: 46 - DNA, 85 bp, Oryza sativa
gaggattggcaaggcatggaagaggggttacctgctgtatggtccaccagggactggcaagtccagcttgattgcagccatggcg

SEQ ID NO: 47 - DNA, 86 bp, Oryza sativa
gaggattggcaaggcatggaagaggggttacctgctagtatggtccaccagggactggcaagtccagcttgattgcagccatggcg

SEQ ID NO: 48 - DNA, 86 bp, Oryza sativa
gaggattggcaaggcatggaagaggggttacctgctggtatggtccaccagggactggcaagtccagcttgattgcagccatggcg

SEQ ID NO: 49 - DNA, 81 bp, Oryza sativa
gaggattggcaaggcatggaagaggggttacctgcttgtccaccagggactggcaagtccagcttgattgcagccatggcg

SEQ ID NO: 50 - DNA, 85 bp, Oryza sativa; identical to SEQ ID NO: 46

SEQ ID NO: 51 - DNA, 83 bp, Oryza sativa
gaggattggcaaggcatggaagaggggttcatcaagctggaccaccagggactggcaagtccagcttgattgcagccatggcg

SEQ ID NO: 52 - DNA, 80 bp, Oryza sativa
gaggattggcaaggcatggaagaggggttacctgctgtccaccagggactggcaagtccagcttgattgcagccatggcg

SEQ ID NO: 53 - DNA, 78 bp, Oryza sativa
gaggattggcaaggcatggaagaggggttacctgctccaccagggactggcaagtccagcttgattgcagccatggcg

SEQ ID NO: 54 - DNA, 77 bp, Oryza sativa
gaggattggcaaggcatggaagaggggttacctgtccaccagggactggcaagtccagcttgattgcagccatggcg

SEQ ID NO: 55 - DNA, 91 bp, Oryza sativa
gaggattggcaaggcatggaagaggggttgattactacaagagaatggtccaccagggactggcaagtccagcttgattgcagccatggcg

SEQ ID NO: 56 - DNA, 84 bp, Oryza sativa
gaggattggcaaggcatggaagaggggttacctgcttatggtccaccagggactggcaagtccagcttgattgcagccatggcg

SEQ ID NO: 57 - DNA, 78 bp, Oryza sativa
gaggattggcaaggcatggaagaggggttaccttgtccaccagggactggcaagtccagcttgattgcagccatggcg

SEQ ID NO: 58 - DNA, 81 bp, Oryza sativa
gaggattggcaaggcatggaagaggggttacctgctggtccaccagggactggcaagtccagcttgattgcagccatggcg

SEQ ID NO: 59 - DNA, 85 bp, Oryza sativa
gaggattggcaaggcatggaagaggggttacctgcagtatggtccaccagggactggcaagtccagcttgattgcagccatggcg

SEQ ID NO: 60 - DNA, 70 bp, Oryza sativa
gaggattggcaaggcatggaagaggggtccaccagggactggcaagtccagcttgattgcagccatggcg

SEQ ID NO: 61 - DNA, 19 bp, Oryza sativa
gaggattgcagccatggcg

SEQ ID NO: 62 - DNA, 85 bp, Oryza sativa
aagaggattggcaaggcatggaagaggggttacctgctgtatggtccaccagggactggcaagtccagcttgattgcagccatgg

SEQ ID NO: 63 - DNA, 86 bp, Oryza sativa
aagaggattggcaaggcatggaagaggggttacctgctagtatggtccaccagggactggcaagtccagcttgattgcagccatgg

SEQ ID NO: 64 - DNA, 86 bp, Oryza sativa
aagaggattggcaaggcatggaagaggggttacctgcttgtatggtccaccagggactggcaagtccagcttgattgcagccatgg

SEQ ID NO: 65 - DNA, 81 bp, Oryza sativa
aagaggattggcaaggcatggaagaggggttacctgctggtccaccagggactggcaagtccagcttgattgcagccatgg

SEQ ID NO: 66 - DNA, 45 bp, Oryza sativa
aagaggattggcaaggcatggaagaggggttacctgcagccatgg

SEQ ID NO: 67 - DNA, 80 bp, Oryza sativa
aagaggattggcaaggcatggaagaggggttacctgctgtccaccagggactggcaagtccagcttgattgcagccatgg

SEQ ID NO: 68 - DNA, 24 bp, Oryza sativa
aagaggattggcaaggcattgacc

SEQ ID NO: 69 - DNA, 45 bp, Oryza sativa; identical to SEQ ID NO: 66